(19) United States
(12) Patent Application Publication
(10) Pub. No.: US 2023/0001098 A1
(43) Pub. Date: Jan. 5, 2023

(54) MEDICATION DELIVERY DEVICE WITH SENSING SYSTEM

(71) Applicant: Eli Lilly and Company, Indianapolis, IN (US)

(72) Inventors: Jay Mark FRIEDLANDER, Carmel, IN (US); Patrick Kevin MURPHY, Allston, MA (US); Sean Matthew PSZENNY, Cambridge, MA (US); Labi SHRESTHA, Somerville, MA (US)

(21) Appl. No.: 17/779,463

(22) PCT Filed: Feb. 20, 2020
(86) PCT No.: PCT/US2020/018962

§ 371 Date: May 24, 2022

Related U.S. Application Data
(60) Provisional application No. 62/811,228, filed on Feb. 27, 2019.
(60) Provisional application No. 62/946,628, filed on Dec. 11, 2019.

(51) Int. Cl. A61M 5/315

(52) U.S. Cl. CPC A61M 5/31568; A61M 5/31553; A61M 5/31593; A61M 2205/3584; A61M 2205/52; A61M 2205/6072

(57) ABSTRACT

Medication delivery devices are provided having a housing comprising a reservoir sized sufficiently to hold medication, a dose button being rotatable relative to the housing to select a dose size of the medication for an injection, a printed circuit board, a switch mounted to the printed circuit board, and a controller in communication with the switch. The controller is configured to generate a first count of a set of signals using a first counting technique, generate a second count of the set of signals using a second counting technique, and determine, based on the first and second count, reliability data of the first and/or second count. The controller can connect, using a wireless communication module, to a remote computing device to wirelessly communicate with the remote computing device over an unencrypted wireless communication channel, and transmit, using the wireless communication module, encrypted data to the remote computing device.

### BACKGROUND

Patients suffering from various diseases must frequently inject themselves with medication. To allow a person to conveniently and accurately self-administer medicine, a variety of devices broadly known as pen injectors or injection pens have been developed. Generally, these pens are equipped with a cartridge including a piston and containing a multi-dose quantity of liquid medication. A drive member is movable forward to advance the piston in the cartridge to dispense the contained medication from an outlet at the distal cartridge end, typically through a needle.

In disposable or prefilled pens, after a pen has been utilized to exhaust the supply of medication within the cartridge, a user discards the entire pen and begins using a new replacement pen. In reusable pens, after a pen has been utilized to exhaust the supply of medication within the cartridge, the pen is disassembled to allow replacement of the spent cartridge with a fresh cartridge, and then the pen is reassembled for its subsequent use.

Such devices may have components that physically interact with one another to result in a state change or an action by the device. For example, the device may have a cap that is removed prior to delivery, a dose button that may be rotated to set a dose and/or actuated to deliver a dose, an “on” button that wakes the device, and so on.

The inventors have appreciated that switches can be used to detect the occurrence of such interactions. The inventors have also appreciated that some of these physical interactions may happen repeatedly. The inventors have thus recognized a need for a simple, low-cost switch that can be repeatedly opened and closed to detect such interactions.

### SUMMARY

The present disclosure relates to a medication delivery device provided, including a housing comprising a reservoir sized sufficiently to hold medication, a dose button being rotatable relative to the housing to select a dose size of the medication for an injection, a printed circuit board, a switch mounted to the printed circuit board, and a controller in communication with the switch. The controller is configured to receive a set of signals from the switch, generate, based on the set of signals, a first count of the set of signals using a first counting technique, generate, based on the set of signals, a second count of the set of signals using a second counting technique, and determine, based on the first count and the second count, data indicative of a reliability of the first count, the second count, or both.

In one embodiment, a computerized method is provided, the method being executed by a controller of a medication delivery device, wherein the controller is in communication with a switch mounted to a printed circuit board, the method comprising receiving a set of signals from the switch, generating, based on the set of signals, a first count of the set of signals using a first counting technique, generating, based on the set of signals, a second count of the set of signals using a second counting technique, and determining, based on the first count and the second count, data indicative of a reliability of the first count, the second count, or both.

In one embodiment, a medication delivery device is provided, including a housing comprising a reservoir sized sufficiently to hold medication, a dose button being rotatable relative to the housing to select a dose size of the medication for an injection, a wireless communication module, and a controller in communication with the wireless communication module. The controller is configured to generate injection event information for the medication delivery device, connect, using the wireless communication module, to a remote computing device to wirelessly communicate with the remote computing device over an unencrypted wireless communication channel, and transmit, using the wireless communication module, encrypted data to the remote computing device over the unencrypted wireless communication channel, wherein the encrypted data comprises data indicative of the injection event information.

In one embodiment, a computerized method is provided, the method being executed by a controller of a medication delivery device, wherein the controller is in communication with a wireless communication module, the method comprising generating injection event information for the medication delivery device, connecting, using the wireless communication module, to a remote computing device to wirelessly communicate with the remote computing device over an unencrypted wireless communication channel, and transmitting, using the wireless communication module, encrypted data to the remote computing device over the unencrypted wireless communication channel wherein the encrypted data comprises data indicative of the injection event information.

In one embodiment, a computing device is provided, including at least one processor in communication with a memory configured to store machine-readable instructions that cause the at least one processor to connect to a wireless communication module of a medication delivery device to wirelessly communicate with the medication delivery device over an unencrypted wireless communication channel, wherein the medication delivery device comprises (a) a housing comprising a reservoir sized sufficiently to hold medication, (b) a dose button being rotatable relative to the housing to select a dose size of the medication for an injection, and (c) a controller in communication with the wireless communication module, and receive, from the medication delivery device using the wireless communication channel, encrypted data over the unencrypted wireless communication channel, wherein the encrypted data comprises injection event information generated by the medication delivery device.

### BRIEF DESCRIPTION OF THE DRAWINGS

Additional embodiments of the disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a perspective view of a medication delivery device having a dose detection system according to aspects of the present disclosure.

FIG. 2 is a partially exploded perspective view of the medication delivery device of FIG. 1, showing a dose button having a support and a cover, where the cover is shown separated from the support.

FIG. 3 is a partially exploded perspective view of the medication delivery device of FIG. 1 showing the components of the dose detection system.

FIG. 4 is a cross-sectional view of the medication delivery device of FIG. 1.

FIG. 5 is a partial cutaway view of a proximal end of the medication delivery device of FIG. 1, showing components of the dose detection system.

FIG. 6 is an underside view of a portion of the dose button of FIG. 1, showing a printed circuit board held within the dose button cover.

FIG. 7 is an exploded view of the portion of the dose button shown in FIG. 6.

FIG. 8 is a perspective view of a flange of a dose detection system of a medication delivery device.

FIG. 9 is a top down view of the flange of FIG. 8.

FIG. 10 is a perspective view of a dose button support.

FIG. 11 is a top down view of the dose button support of FIG. 10.

FIG. 12 is a perspective view of a printed circuit board and sensor switch according to aspects of the present disclosure.

FIG. 13 is a perspective view of a cantilevered arm and base of the sensor switch of FIG. 12.

FIG. 14 is a side view of the cantilevered arm and base of FIG. 13.

FIG. 15 is a side view of the cantilevered arm of FIG. 12 positioned between two teeth of a flange.

FIG. 16 shows the cantilevered arm of FIG. 15 being pushed by one of the teeth of the flange during rotation of the flange.

FIG. 17 shows the cantilevered arm being pushed further by the tooth of the flange such that a portion of the cantilevered arm has moved toward and is in contact with a conductive pad, closing the switch.

FIG. 18 shows the cantilevered arm sliding over the tooth of the flange.

FIG. 19 shows the cantilevered arm interacting with the next adjacent tooth of the flange.

FIG. 20 is a perspective view of a switch design according to another embodiment.

FIG. 21 is a side view of the switch of FIG. 20.

FIG. 22 is a perspective view of a switch design according to another embodiment.

FIG. 23 is a side view of the switch of FIG. 22.

FIG. 24 is a perspective view of the switch of FIG. 22 interacting with a rotating flange.

FIG. 25 is a side view of the switch of FIG. 20 that shows a center of mass of the switch.

FIG. 26A is an exemplary block diagram illustrating functional aspects of a printed circuit board for processing signals from a sensor, according to some embodiments.

FIG. 26B is a graph showing an exemplary set of signals generated by a sensor, according to some embodiments.

FIG. 27 is a flow chart showing an exemplary computerized method for processing signals from a rotational sensor, according to some embodiments.

FIG. 28 is a flow chart showing an exemplary encryption scheme that uses an open communication channel to exchange encrypted information and accesses encryption information from a cloud computing device, according to some embodiments.

FIG. 29 is a flow chart showing an exemplary encryption scheme that uses an open communication channel to exchange encrypted information and determines encryption information from a barcode on the medication delivery device, according to some embodiments.

FIG. 30 is a diagram illustrating various states and associated functionality of a medication delivery device, according to some embodiments.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

The present disclosure relates to sensing systems for medication delivery devices.

In one aspect, the sensing system includes a switch with an arm that is moveable to open and close the switch. The switch is used for sensing of relative rotational movement between a dose-setting assembly and an actuator of the medication delivery device in order to determine the amount of a dose delivered by a medication delivery device. The sensed relative rotational movements are correlated to the amount of the dose delivered. By way of illustration, the medication delivery device is described in the form of a pen injector. However, the medication delivery device may be any device which is used to set and to deliver a dose of a medication, such as pen injectors, infusion pumps and syringes. The medication may be any of a type that may be delivered by such a medication delivery device.

The present disclosure also relates to various software and/or hardware-based techniques of the medication delivery device and a remote computing device that is in communication with the medication delivery device.

In one aspect, the medication delivery device includes a housing with a reservoir sized sufficiently to hold medication, a dose button being rotatable relative to the housing to select a dose size of the medication for an injection, a printed circuit board, a switch mounted to the printed circuit board, and a controller in communication with the switch. The controller is configured to receive a set of signals from the switch, generate, based on the set of signals, a first count of the set of signals using a first counting technique and a second count of the set of signals using a second counting technique, and determine, based on the first count and the second count, data indicative of a reliability of the first count, the second count, or both.

In another aspect, the medication delivery device includes a housing with a reservoir sized sufficiently to hold medication, a dose button being rotatable relative to the housing to select a dose size of the medication for an injection, a wireless communication module, and a controller in communication with the wireless communication module. The controller is configured to generate injection event information for the medication delivery device, connect, using the wireless communication module, to a remote computing device to wirelessly communicate with the remote computing device over an unencrypted wireless communication channel, and transmit, using the wireless communication module, encrypted data to the remote computing device over the unencrypted wireless communication channel.

Devices described herein may further comprise a medication, such as for example, within a reservoir or cartridge 20. In another embodiment, a system may comprise one or more devices including device 10 and a medication. The term “medication” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies and any therapeutic agent that is capable of delivery by the above device. The medication as used in the device may be formulated with one or more excipients. The device is operated in a manner generally as described above by a patient, caregiver or healthcare professional to deliver medication to a person.

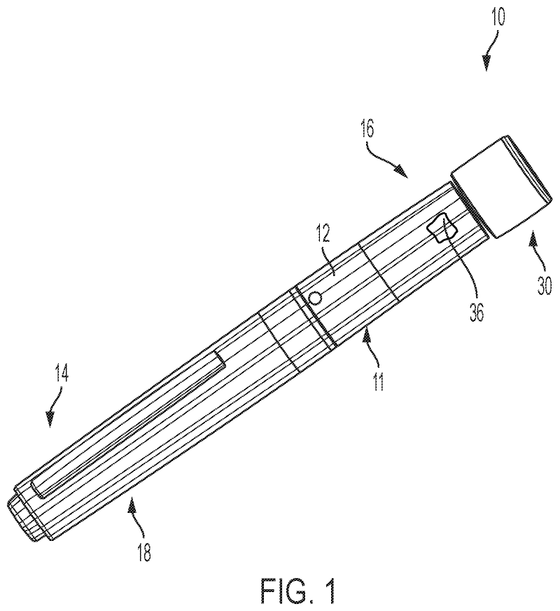

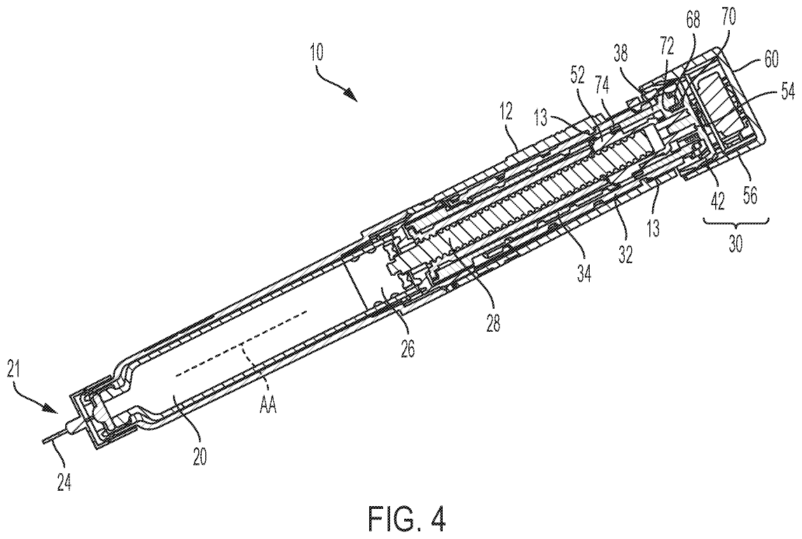

An exemplary medication delivery device 10 is illustrated in FIGS. 1-4 as a pen injector configured to inject a medication into a patient through a needle. Device 10 includes a body 11 that may comprise an elongated, pen-shaped housing 12 including a distal portion 14 and a proximal portion 16. Distal portion 14 may be received within a pen cap 18. Referring to FIG. 4, distal portion 14 may contain a reservoir or cartridge 20 configured to hold the medicinal fluid to be dispensed through the outlet 21 of the housing a dispensing operation. The outlet 21 of distal portion 14 may be equipped with an injection needle 24. In some embodiments, the injection needle is removable from the housing. In some embodiments, the injection needle is replaced with a new injection needle after each use.

A piston 26 may be positioned in reservoir 20. The medication delivery device may include an injecting mechanism positioned in proximal portion 16 that is operative to advance piston 26 toward the outlet of reservoir 20 during the dose dispensing operation to force the contained medicine through the needled end. The injecting mechanism may include a drive member 28, illustratively in the form of a screw, that is axially moveable relative to housing 12 to advance piston 26 through reservoir 20.

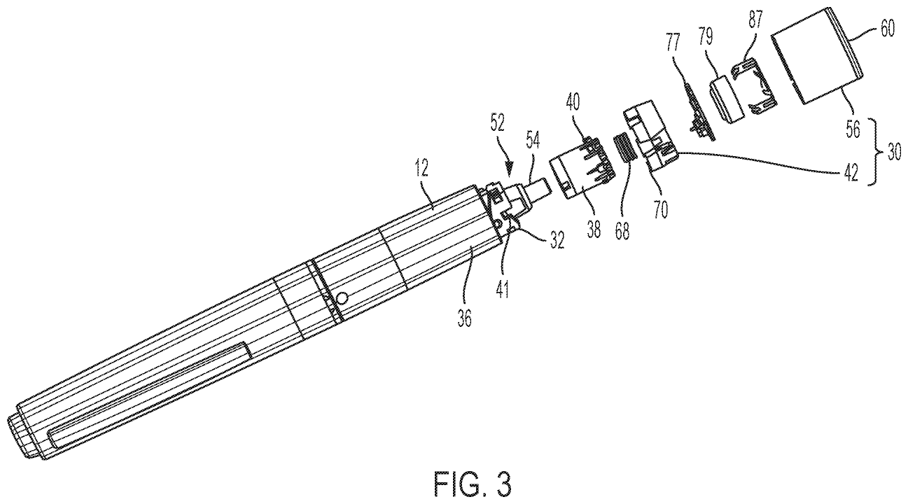

The device may include a dose-setting assembly coupled to the housing 12 for setting a dose amount to be dispensed by device 10. As best seen in FIGS. 3 and 4, in the illustrated embodiment, the dose-setting assembly includes a dose-setting screw 32 and a flange 38. The dose-setting screw 32 is in the form of a screw element operative to spiral (i.e., simultaneously move axially and rotationally) about a longitudinal axis AA of rotation relative to housing 12 during dose setting and dose dispensing. FIGS. 3 and 4 illustrate the dose-setting screw 32 fully screwed into housing 12 at its home or zero dose position. Dose-setting screw 32 is operative to screw out in a proximal direction from housing 12 until it reaches a fully extended position corresponding to a maximum dose deliverable by device 10 in a single injection. The extended positon may be any position between a position corresponding to an incremental extended position (such as a dose setting a 0.5 or 1 unit) to a fully extended position corresponding to a maximum dose deliverable by device 10 in a single injection and to screw into housing 12 in a distal direction until it reaches the home or zero position corresponding to a minimum dose deliverable by device 10 in a single injection.

Referring to FIGS. 3 and 4, dose-setting screw 32 includes a helically threaded outer surface that engages a corresponding threaded inner surface 13 of housing 12 to allow dose-setting screw 32 to spiral (i.e. simultaneously rotate and translate) relative to housing 12. Dose-setting screw 32 further includes a helically threaded inner surface that engages a threaded outer surface of sleeve 34 (FIG. 4) of device 10. The outer surface of dose-setting screw 32 includes dose indicator markings, such as numbers that are visible through a dosage window 36 to indicate to the user the set dose amount.

As mentioned above, in some embodiments, the dose-setting assembly further includes a tubular flange 38 that is coupled in the open proximal end of dose-setting screw 32 and is axially and rotationally locked to the dose-setting screw 32 by protrusions 40 received within openings 41 in the dose-setting screw 32. The protrusions 40 of the flange 38 can be seen in FIGS. 3, 8 and 9, and the openings 41 of the dose-setting screw 32 can be seen in FIG. 3.

As seen in FIGS. 3 and 4, delivery device 10 may include an actuator assembly having a clutch 52 and a dose button 30. The clutch 52 is received within the dose-setting screw 32, and the clutch 52 includes an axially extending stem 54 at its proximal end. The dose button 30 of the actuator assembly is positioned proximally of the dose-setting screw 32 and flange 38. Dose button 30 includes a support 42, also referred to herein as an “under button,” and a cover 56, also referred to herein as an “over button.” As will be discussed, the support 42 and cover 56 enclose electronics components used to store and/or communicate data relating to amount of dose delivered by a medication delivery device.

The support 42 of the dose button may be attached to the stem 54 of the clutch 52, such as with an interference fit or an ultrasonic weld, so as to axially and rotatably fix together dose button 30 and clutch 52.

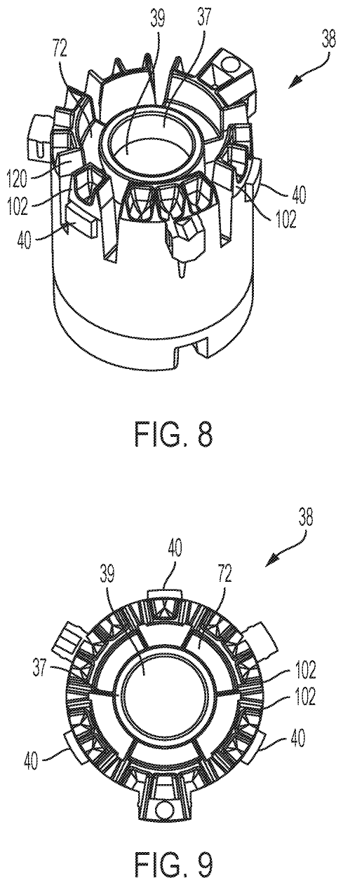

In some embodiments, a portion of the clutch may pass through a lumen 39 of the flange 38. The lumen 39 of the flange is best seen in FIGS. 8 and 9. The lumen 39 may, in some embodiments, serve to help center the clutch 52 in place.

Proximal face 60 of the dose button 30 may serve as a push surface against which a force can be applied manually, i.e., directly by the user to push the actuator assembly (dose button 30 and clutch 52) in a distal direction. A bias member 68, illustratively a spring, may be disposed between the distal surface 70 of support 42 and a proximal surface 72 of tubular flange 38 (FIGS. 8 and 9) to urge the support 42 of the actuation assembly and the flange 38 of the dose-setting assembly axially away from each other. Dose button 30 is depressible by a user to initiate the dose dispensing operation. In some embodiments, the bias member 68 is seated against this proximal surface 72 and may surround a raised collar 37 of the flange 38.

Delivery device 10 is operable in a dose setting mode and a dose dispensing mode. In the dose setting mode of operation, the dose button 30 is rotated relative to housing 12 to set a desired dose to be delivered by device 10. In some embodiments, rotating the dose button 30 in one direction relative to the housing 12 causes the dose button 30 to axially translate proximally relative to the housing 12, and rotating the dose button 30 in the opposite direction relative to the housing 12 causes the dose button 30 to axially translate distally relative to the housing. In some embodiments, clockwise rotation of the dose button moves the dose button 30 distally, and counter-clockwise rotation of the dose button moves the dose button proximally, or vice versa.

In some embodiments, rotating the dose button 30 to axially translate the dose button 30 in the proximal direction serves to increase the set dose, and rotating the dose button 30 to axially translate the dose button 30 in the distal direction serves to decrease the set dose. The dose button 30 is adjustable in pre-defined rotational increments corresponding to the minimum incremental increase or decrease of the set dose during the dose setting operation. The dose button may include a detent mechanism such that each rotational increment produces an audible and/or tactile “click.” For example, one increment or “click” may equal one-half or one unit of medication.

In some embodiments, the set dose amount may be visible to the user via the dial indicator markings shown through a dosage window 36. During the dose setting mode, the actuator assembly, which includes the which includes the dose button 30 and clutch 52, moves axially and rotationally with the dose-setting assembly, which includes the flange 38 and the dose-setting screw 32.

Dose-setting screw 32 and flange 38 are fixed rotationally to one another, and rotate and move proximally during dose setting, due to the threaded connection of the dose-setting screw 32 with housing 12. During this dose setting motion, the dose button 30 is rotationally fixed relative to the flange 38 and the dose-setting screw 32 by complementary splines 74 of flange 38 and clutch 52 (FIG. 4), which are urged together by the bias member 68. In the course of dose setting, the dose-setting screw 32, flange 38, clutch 52, and dose button 30 move relative to the housing 12 in a spiral manner (i.e. simultaneous rotation and axial translation) from a “start” position to an “end” position. This rotation and translation relative to the housing is in proportion to the amount of dose set by operation of the medication delivery device 10.

Once the desired dose is set, device 10 is manipulated so the injection needle 24 properly penetrates, for example, a user's skin. The dose dispensing mode of operation is initiated in response to an axial distal force applied to the proximal face 60 of dose button 30. The axial force is applied by the user directly to dose button 30. This causes axial movement of the actuator assembly (dose button 30 and clutch 52) in the distal direction relative to housing 12.

The axial shifting motion of the actuator assembly compresses biasing member 68 and reduces or closes the gap between dose button 30 and the tubular flange 38. This relative axial movement separates the complementary splines 74 on clutch 52 and flange 38, and thereby disengages the dose button 30 from being rotationally fixed to the flange 38 and the dose-setting screw 32. In particular, the dose-setting screw 32 is rotationally uncoupled from the dose button 30 to allow backdriving rotation of the dose-setting screw 32 relative to the dose button 30 and the housing 12. Also, while the dose-setting screw 32 and flange 38 are free to rotate relative to the housing 12, the dose button 30 is held from rotating relative to the housing 12 by the user's engagement of dose button 30 by pressing against it.

As dose button 30 and clutch 52 are continued to be axially plunged without rotation relative to housing 12, dose-setting screw 32 screws back into housing 12 as it spins relative to dose button 30. The dose markings that indicate the amount still remaining to be injected are visible through window 36. As dose-setting screw 32 screws down distally, drive member 28 is advanced distally to push piston 26 through reservoir 20 and expel medication through needle 24.

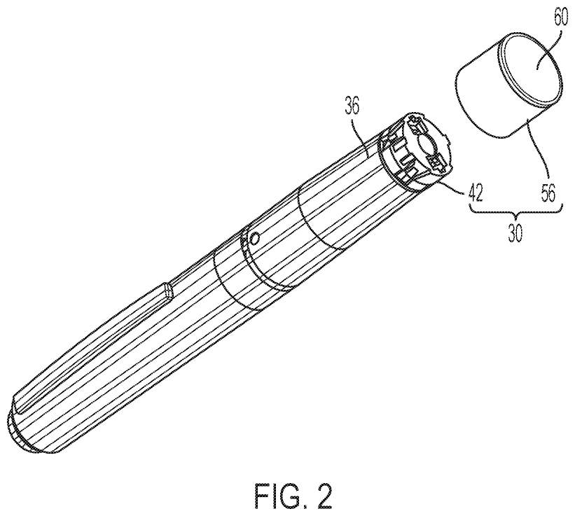

During the dose dispensing operation, the amount of medicine expelled from the medication delivery device is proportional to the amount of rotational movement of the dose-setting screw 32 relative to the housing 12 as the dose-setting screw 32 screws back into housing 12. In some embodiments, because the dose button 30 is rotationally fixed relative to the housing 12 during the dose dispensing mode, the amount of medicine expelled from the medication delivery device may be viewed as being proportional to the amount of rotational movement of the dose-setting screw 32 relative to the dose button 30 as the dose-setting 32 screws back into housing 12. The injection is completed when the internal threading of dose-setting screw 32 has reached the distal end of the corresponding outer threading of sleeve 34 (FIG. 4). Device 10 is then once again arranged in a ready state or zero dose position as shown in FIGS. 2 and 4.

As discussed above, the dose delivered may be derived based on the amount of rotation of the dose-setting assembly (flange 38 and dose-setting screw 32) relative to the actuator assembly (clutch 52 and dose button 30) during dose delivery. This rotation may be determined by detecting the incremental movements of the dose-setting assembly which are “counted” as the dose-setting assembly is rotated during dose delivery.

Further details of the design and operation of an exemplary delivery device 10 may be found in U.S. Pat. No. 7,291,132, entitled Medication Dispensing Apparatus with Triple Screw Threads for Mechanical Advantage, the entire disclosure of which is hereby incorporated by reference herein. Another example of the delivery device is an auto-injector device that may be found in U.S. Pat. No. 8,734,394, entitled “Automatic Injection Device With Delay Mechanism Including Dual Functioning Biasing Member,” which is hereby incorporated by reference in its entirety, where such device being modified with one or more various sensor systems described herein to determine an amount of medication delivered from the medication delivery device based on the sensing of relative rotation within the medication delivery device. Another example of the delivery device is a reusable pen device that may be found in U.S. Pat. No. 7,195,616, entitled “Medication Injector Apparatus with Drive Assembly that Facilitates Reset,” which is hereby incorporated by reference in its entirety, where such device being modified with one or more various sensor systems described herein to determine an amount of medication delivered from the medication delivery device based on the sensing of relative rotation within the medication delivery device.

Described herein is a dose detection system that may be operable to determine the amount of dose delivered based on relative rotation between a dose setting member and the device body. The dose detection system utilizes a dose setting member attached to the device body and rotatable relative to the device body about an axis of rotation during dose delivery. A sensed element is attached to and rotationally fixed with the dose setting member. An actuator is attached to the device body and is held against rotation relative to the device body during dose delivery. The sensed element thereby rotates relative to the actuator during dose delivery in relation to the amount of dose delivered.

In some embodiments, the dose detection system comprises a rotational sensor attached to the actuator assembly and a sensed element that includes surface features that are equally radially spaced about the axis of rotation of the sensed element.

In some embodiments, the dose detection systems may include a sensor and a sensed component attached to components of the medication delivery device. The term “attached” encompasses any manner of securing the position of a component to another component or to a member of the medication delivery device such that they are operable as described herein. For example, a sensor may be attached to a component of the medication delivery device by being directly positioned on, received within, integral with, or otherwise connected to, the component. Connections may include, for example, connections formed by frictional engagement, splines, a snap or press fit, sonic welding or adhesive.

The term “directly attached” is used to describe an attachment in which two components, or a component and a member, are physically secured together with no intermediate member, other than attachment components. An attachment component may comprise a fastener, adapter or other part of a fastening system, such as a compressible membrane interposed between the two components to facilitate the attachment. A “direct attachment” is distinguished from attachment where the components/members are coupled by one or more intermediate functional members.

The term “fixed” is used to denote that an indicated movement either can or cannot occur. For example, a first member is “fixed rotationally” with a second member if the two members are required to move together in rotation. In one aspect, a member may be “fixed” relative to another member functionally, rather than structurally. For example, a member may be pressed against another member such that the frictional engagement between the two members fixes them together rotationally, while the two members may not be fixed together absent the pressing of the first member.

Various sensor arrangements are contemplated herein. In general, the sensor arrangements comprise a sensor and a sensed component. The term “sensor” refers to any component which is able to detect the relative position or movement of the sensed component. The sensor may be used with associated electrical components to operate the sensor. The “sensed component” is any component for which the sensor is able to detect the position and/or movement of the sensed component relative to the sensor. For the dose detection system, the sensed component rotates relative to the sensor, which is able to detect the rotational movement of the sensed component. The sensor may comprise one or more sensing elements, and the sensed component may comprise one or more sensed elements. The sensor detects the movement of the sensed component and provides outputs representative of the movement of the sensed component.

Illustratively, the dose detection system includes an electronics assembly suitable for operation of the sensor arrangement as described herein. The medication delivery device may include a controller that is operably connected to the sensor to receive outputs from the sensor. The controller begins receiving generated signals from the sensor indicative of counts from first to last one for a total number of counts that is used for determining total displacement, e.g. angular displacement. In the case of detecting an angular movement of a dose-setting assembly, the controller may be configured to receive data indicative of the angular movement of the dose-setting assembly that can be used to determine from the outputs the amount of dose delivered by operation of the medication delivery device. The controller may be configured to determine from the outputs the amount of dose delivered by operation of the medication delivery device. The controller may include conventional components such as a processor, power supply, memory, microcontrollers, etc. Alternatively, at least some components may be provided separately, such as by means of a computer, smart phone or other device. Means are then provided to operably connect the external controller components with the sensor at appropriate times, such as by a wired or wireless connection.

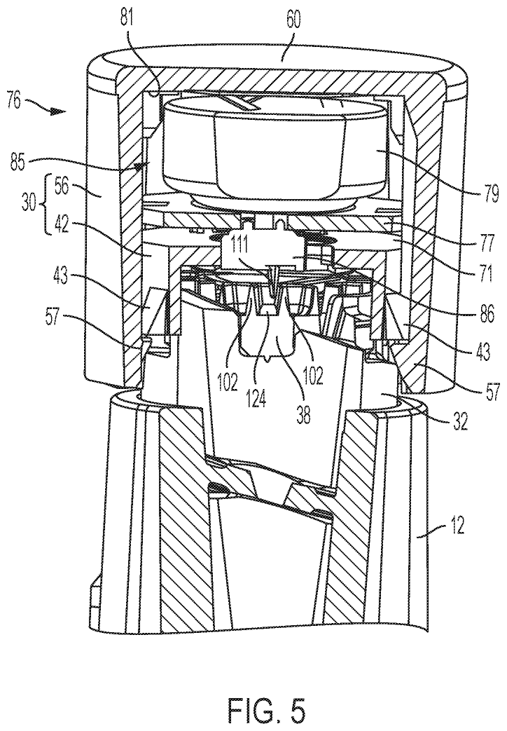

According to one aspect, the electronics assembly includes a sensor arrangement including one or more sensors operatively communicating with a processor for receiving signals from the sensor representative of the sensed rotation. An exemplary electronics assembly 76 is shown in FIGS. 5-7 and can include a sensor 86, and a printed circuit board (PCB) 77 having a plurality of electronic components. The printed circuit board may be a flexible printed circuit board. The circuit board of the electronics assembly 76 may include a microcontroller unit (MCU) as the controller comprising at least one processing core and internal memory. The electronics assembly may include a power source 79, e.g. a battery, illustratively a coin cell battery, for powering the components. The controller of electronics assembly 76 may include control logic operative to perform the operations described herein, including detecting the angular movement of the dose-setting assembly during dose setting and/or dose delivery and/or detecting a dose delivered by medication delivery device 10 based on a detected rotation of the dose-setting assembly relative to the actuator assembly. Many, if not all of the components of the electronics assembly, may be contained in a compartment 85 within the dose button 30. In some embodiments, the compartment 85 may be defined between a proximal surface 71 of support 42 of the dose button and a distal surface 81 of the cover 56 of the dose button. In the embodiment shown in FIG. 5, the electronics assembly 76 is permanently integrated within the dose button 30 of the delivery device. In other embodiments, the electronics assembly is provided as a module that can be removably attached to the actuator assembly of the medication delivery device.

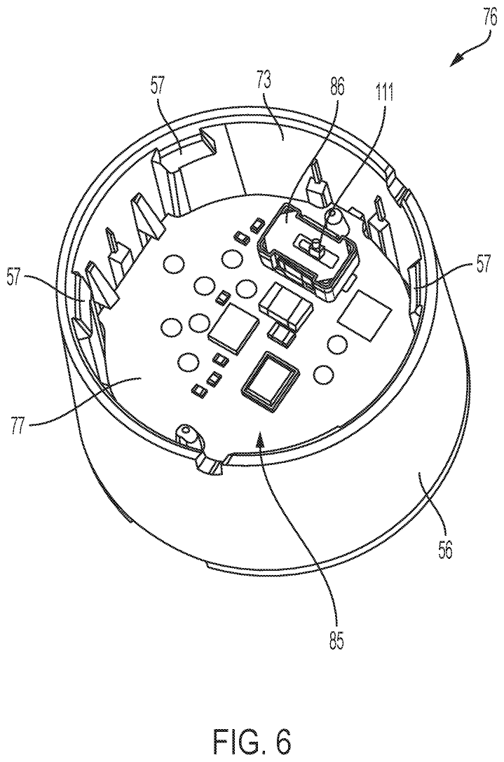

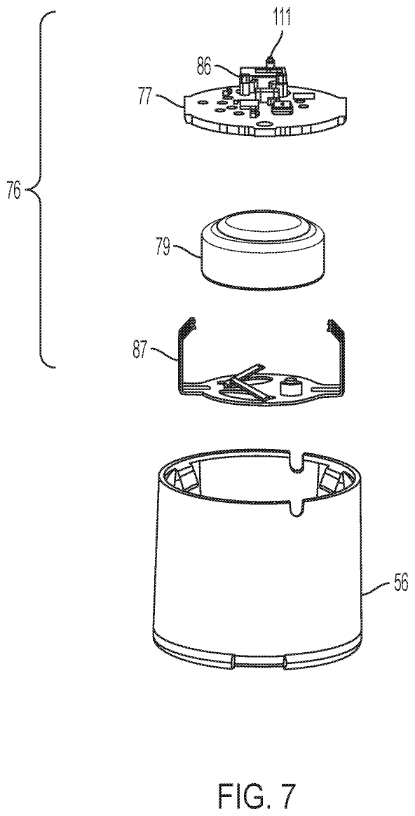

An underside view of the electronics assembly 76 held within the cover 56 is shown in FIG. 6, and an exploded view of the electronics assembly 76 is shown in FIG. 7. As shown in FIGS. 6 and 7, the electronics assembly 76 may include a printed circuit board (PCB) 77 and a sensor 86 having a contact surface 111. As shown in FIG. 7, the electronics assembly 76 may also include a battery 79 and a battery cage 87.

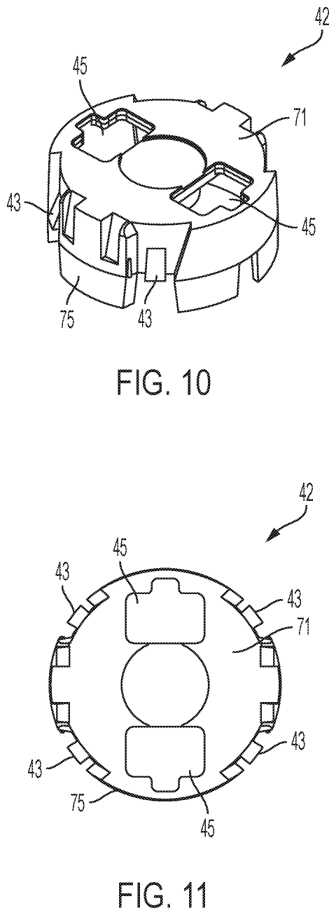

In some embodiments, at least a portion of the sensor 86 extends out of the compartment 85 of the dose button 30. As best seen in FIGS. 10 and 11, the support 42 of the dose button 30 may include one or more openings 45 through which the sensor 86 can extend through. In some embodiments, during assembly of the medication delivery device, the contact surface 111 of the sensor 86 is passed through the opening 45 of the support 42. This may permit the contact surface 111 of the sensor to interact with a component that is external to the compartment 85 of the dose button 30. In some embodiments, while only one of the openings 45 in the support 42 is needed to accommodate a sensor, a second opening may be provided, e.g. for symmetry of the support component, which help with manufacturing of the component and/or assembly of the component with the medication delivery device.

The controller of electronics assembly 76 may be operative to store the total angular movement used for determining dose delivery and/or the detected dose delivery in local memory (e.g., internal flash memory or on-board EEPROM). The controller may be further operative to wirelessly transmit a signal representative of the total counts, total angular movement, and/or detected dose to an external device, such as a user's mobile device or a remote server. Transmission may, for example, be over a Bluetooth low energy (BLE) or other suitable short or long range wireless communication protocol. Illustratively, the BLE control logic and controller are integrated on the same circuit.

As discussed, according to one aspect, the dose detection system involves detecting relative rotational movement between two assemblies of the medication delivery device. With the extent of rotation having a known relationship to the amount of a delivered dose, the sensor operates to detect the amount of angular movement from the start of a dose injection to the end of the dose injection. For example, in some embodiments, the relationship for a pen injector is that an angular displacement of a dose-setting assembly of 18° is the equivalent of one unit of dose, although other angular relationships are also suitable, such as, for example, 9, 10, 15, 20, 24 or 36 degrees may be used for a unit or a half unit. The sensor system is operable to determine the total angular displacement of a dose setting member during dose delivery. Thus, if the angular displacement is 90°, then 5 units of dose have been delivered.

The angular displacement is determined by counting increments of dose amounts as the injection proceeds. For example, a sensing system may use a repeating pattern of a sensed element, such that each repetition is an indication of a predetermined degree of angular rotation. Conveniently, the pattern may be established such that each repetition corresponds to the minimum increment of dose that can be set with the medication delivery device.

The dose detection system components may be permanently or removably attached to the medication delivery device. In some embodiments, at least some of the dose detection system components are provided in the form of a module that is removably attached to the medication delivery device. In other embodiments, the dose detection system components are permanently attached to the medication delivery device.

In some embodiments, a sensor may detect, during dose delivery, the relative rotation of a sensed component that is rotationally fixed to the dose-setting screw 32, from which is determined the amount of a dose delivered by the medication delivery device. In an illustrative embodiment, a rotational sensor is attached, and rotationally fixed, to the actuator assembly. The actuator assembly does not rotate relative to the device housing during dose delivery.

In some embodiments, a sensed component is attached, and rotationally fixed, to the dose-setting screw 32, which rotates relative to the dose button 30 and the device housing 12 during dose delivery. In some of the embodiments described herein, the sensed component includes a ring structure having a plurality of proximally extending projections circumferentially disposed relative to one another. Projections are shaped and sized to deflect a movable element of the rotational sensor. One illustrative embodiment of such a sensed component is tubular flange 38, best seen in FIGS. 3, 5, 8, and 9. Embodiments described herein may be provided for a module that is removably attachable to the dose button of the delivery device or integrated within the dose button of the delivery device.

During dose delivery, dose-setting screw 32 is free to rotate relative to dose button 30. In the illustrative embodiment, the electronics assembly 76 is rotationally fixed with the dose button 30 and does not rotate during dose delivery.

As seen in FIGS. 2, 3 and 5, the dose button 30 comprises a cover 56 coupled to a support 42. An electronics assembly 76 may be at least partially contained within a compartment 85 defined between the cover 56 and the support. In some embodiments, the cover and support have corresponding splines that engage with one another to couple the cover and support together. For example, in some embodiments, the cover 56 may couple to the support 42 via one or more snaps 57 on the cover 56 and corresponding to one or more protrusions 43 on the support. As seen in FIGS. 5 and 6, the snaps 57 on the cover 56 may be directed radially inwardly from an inner circumferential sidewall 73. As seen in FIGS. 5, 10 and 11, the protrusions 43 on the support 42 may be directed radially outwardly from an outer circumferential sidewall 75 of the support 42. The protrusions 43 may form a triangular ramp shape.

The snaps 57 on the cover 56 are configured to snap over and mate with the protrusions 43 on the support to couple the cover to the support. In some embodiments, the protrusion on the support comprises a continuous annular protrusion around the outer circumferential sidewall of the support. The cover 56 may attach to the support 42 via frictional engagement, interference fit or any other suitable fit. In some embodiments, the cover 56 is permanently fixed to the support 42 during assembly, e.g. via ultrasonic welding, adhesive, or other suitable fixation approach.

As seen in FIGS. 8 and 9, the tubular flange 38 may include a plurality of axially directed teeth 102 that are equally radially spaced about a rotation axis and arranged to correlate to the equivalent of one unit of dose. In this illustrative embodiment, the tubular flange 38 includes 20 teeth 102 that are equally rotationally spaced from one another, such that the rotation distance between two adjacent teeth corresponds to 18 degrees of rotation. Thus, with the tubular flange 38 of FIG. 8, 18 degrees of rotation of the tubular flange 38 may be used to represent one dosage unit or a half dosage unit. It should be appreciated that, in other embodiments, different total numbers of teeth may be used to create other angular relationships, such as, for example, 9, 10, 15, 18, 20, 24 or 36 degrees may be used for a unit or 0.5 unit.

A recess 124 may be defined between each pair of adjacent teeth 102. Each tooth 102 may have an approximately triangular shaped profile, each having a surface 120 against which a contact surface 111 of a sensor may slide.

In some embodiments, the sensor for detecting rotation of the tubular flange includes a movable element that has a contact portion capable of resting against the teeth of the tubular flange and is spring-biased such that the contact surface is configured to slide against and over the teeth during rotation of the flange relative to the actuator assembly during dose delivery. The sensor is responsive to the movement of the contact portion over the teeth and generates signals corresponding to the flange. A controller is responsive to the signals generated by the sensor to determine a dose count for determining the dosage delivered based on the detected rotation of the flange relative to the actuator assembly during dose delivery.

The contact surface may be biased against the physical features of the tubular flange to ensure proper contact between the contact surface and the physical features during rotation. In one embodiment, the movable element is a resilient member having one portion attached to the actuator at a location displaced from the contact surface. In one example, the movable element is a following member comprising a beam attached at one end to the actuator and having the contact surface at the other end. The beam is flexed to urge the contact surface in the direction of the surface features. Alternatively, the movable element may be biased in any of a variety of other ways. In addition to the use of a resilient beam, the biasing may be provided, for example, by use of a spring component. Such spring component may for example comprise a compression, tension, or torsion coil spring. In yet other embodiments, the movable element may be biased against the surface features of the sensed element by a separate resilient member or spring component bearing against the movable element.

FIG. 5 depicts an illustrative embodiment of a sensor 86 having a contact surface 111 interacting with teeth 102 of a tubular flange 38. As the flange 38 rotates relative to the dose button 30 during delivery, the teeth 102 of the flange contact and slide against the contact surface 111 of the sensor 86, causing the contact surface 111 to move in an oscillating manner. The movement of the contact surface 111 may be a combination of axial and lateral movement as the contact surface 111 slides into and out of the recesses 124 defined between the teeth 102 of the flange 38. The sensor 86 may be configured to track the movement of the contact surface 111 and associate the movement with an output signal that is sent to a controller. In one embodiment, the sensor 86 is a microswitch, where the contact surface in comprises a trigger arm extending from a sensor body, which is capable of moving between a neutral position and an activated position for signalling a change in state electronically, usually between the open and closed state, used to determine data. In the neutral position, the arm may be biased at a position (generally orthogonal to the body as shown in FIG. 5). In the activated position, the arm is moved from the neutral position by some distance or angle, and after the passage of the tooth, the switch returns to is neutral position. As described later, the contact surface in may include a differently configured arm and sensor system. All the sensors described herein may be in the open state when not activated and in the closed state when activated, or vice versa, that is, may be in the closed state when not activated and in the open state when activated.

As alternative to teeth on the tubular flange, surface features that interact with the sensor may comprise anything detectable by the sensor. The sensor arrangement may be based on a variety of sensed characteristics, including tactile, optical, electrical and magnetic properties, for example. In the illustrative embodiments shown in the figures, the surface features are physical features which allow for detection of incremental movements as the dose-setting assembly rotates relative to the actuator assembly. In alternative embodiments, the sensor may be a piezoelectric sensor, a magnetic sensor such as a Hall effect sensor, an accelerometer for detecting vibration, e.g. of a ratcheting or other detent mechanism, where vibration can be correlated with rotational movement, an optical sensor such as a reflective sensor, an interrupter sensor, or an optical encoder, or any other sensor suitable for sensing rotation of a first component relative to a second component.

In some embodiments, when a user presses axially on face 60 of the dose button 30, the dose button 30 advances distally relative to the housing 12, compressing spring 68. Continued pressing of the dose button 30 distally results in back driving of the dose-setting screw 32 in a spiral direction relative to housing 12. As a result, the dose-setting screw 32 and flange 38 are driven to rotate by the axially pressing upon the dose button 30. In some embodiments, the dose detection system is operable for dose detection only while the dose button is being pressed.

In some embodiments, the electronics assembly may include a clock or timer to determine the time elapsed between counts caused by trigger of the rotational sensor from the surface features of the sensed element. When no counts have been detected by the controller after a period of time this may be used to indicate that the dose has completed.

In some embodiments, a single sensing system may be employed for both dose detection sensing and wake-up activation. For example, upon the initial sensing of rotation of the sensed element by the sensor, the controller is configured to allow wake-up or activation of the electronics assembly to a greater or full power state. The wake-up feature is configured to allow power transmission from the power source (shown as battery) for powering up the electronic components for dose sensing in order to minimize inadvertent power loss or usage when a dose dispensing event is not occurring. In other embodiments, a separate wake-up switch may be provided and arranged within the dose button housing and triggered when the dose button is in its distal position. After activation of the electronics assembly, the controller begins receiving generated signals from the rotational sensor indicative of counts from first to last one for a total number of counts that is used for determining total angular displacement and thus the amount of dose delivered.

In some embodiments, the electronics assembly may have a controller that is configured to receive an output signal from a rotational sensor. The controller of the electronics assembly may be programmed to convert the intermediate signal to a conditioned digital signal, which may be a single step/square wave with a predetermined width representing a predetermined time. In some embodiments, output signals that are less than a predetermined level may be filtered out and ignored.

According to one aspect, a medication delivery device includes a repeatedly activatable switch that may serve as a sensor. In some embodiments, the switch serves as the rotational sensor in the dose detection system described above. In other embodiments, however, the switch may be used to detect other activity such as removal of a cap.

In some embodiments, the switch comprises a conductive pad and a cantilevered arm that is moveable relative to the conductive pad. The cantilevered arm may be mounted to a printed circuit board at a first end, and a second end of the arm may be unattached and free to move relative to the printed circuit board.

According to one aspect, the switch may have one or more features that help to permit the switch to repeatedly open and close. The switch may have one or more features that help the switch to avoid plastic deformation during repeated opening and closing, thus helping to maintain durability of the switch.

In some embodiments, at the first end of the cantilevered arm, where the arm attaches to the printed circuit board, the arm has a first curved portion. During switch closure, this first curved portion may be moved toward a straight configuration, and during switch opening, the straight configuration may be moved back toward the curved configuration. In the unstressed state, this first portion may be biased toward the curved configuration. Accordingly, the cantilevered arm may act as a spring that stores potential energy as it is moved during sliding interaction against a sensed component, where the stored potential energy is released to move the arm back toward the unstressed state when the sliding contact force against the arm has decreased.

In some embodiments, the cantilevered arm may transition from the first curved portion to a second curved portion that is configured to move toward and contact the conductive pad that is mounted to the PCB. Contact between the second curved portion and the conductive pad closes the switch, while lack of contact between the second curved portion and the conductive pad opens the switch.

In some embodiments, the cantilevered arm may include a third curved portion configured to contact and slide against the sensed component, e.g. against the teeth of a rotating tubular flange 38 shown in FIGS. 8 and 9. In some embodiments, the third curved portion connects the first curved portion to the second curved portion. In such embodiments, the second curved portion, which is configured to contact the conductive pad, may be located at the second end of the cantilevered arm, i.e. where the cantilevered arm truncates. In other embodiments, the second curved portion connects the first and third curved portions. In such embodiments, the third curved portion, which is configured to contact the sensed component, may be located at the second end of the cantilevered arm, i.e. where the cantilevered arm truncates.

In some embodiments, having the curved portions of the cantilevered arm may help to avoid high strain concentrations in the arm, and may thus help to prevent plastic deformation of the arm. However, it should be appreciated that one or more of the curved portions may be shaped differently in other embodiments.

In some embodiments, the switch may have one or more features that help the switch to provide a cleaner, more easily readable output signal such that a controller can more accurately identify when the switch is open and when the switch is closed. In some embodiments, a blocking protrusion may be provided to interact with the second curved portion of the cantilevered arm as the arm makes contact with the conductive pad. This blocking protrusion may be situated directly adjacent to or near the conductive pad and may prevent the arm from moving past the conductive pad. In some embodiments, the presence of the blocking protrusion may help to reduce “bounce” of the cantilevered arm that may aid in producing a cleaner output signal from the switch. In some cases, “bounce” from the cantilevered arm may cause the arm to rapidly and repeatedly contact and separate from the conductive pad in a short period of time, which can create a noisy output signal that may be difficult for a controller to interpret. The blocking protrusion may help to provide sustained contact between the cantilevered arm and the conductive pad to provide a cleaner output signal. In some embodiments, the blocking protrusion may be made from a shock-absorbing material that may help to deaden the impact of the cantilevered arm against the blocking protrusion, in order to decrease bounce or other vibration.

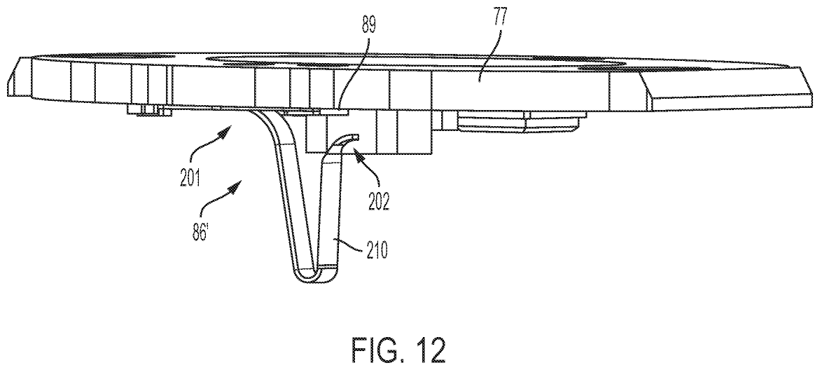

One illustrative example of a switch is shown in FIG. 12, which depicts a switch 86′ having a conductive pad 89 and the contact surface comprises a cantilevered arm 210. The conductive pad 89 and a first end 201 of the cantilevered arm 210 are mounted to a PCB 77.

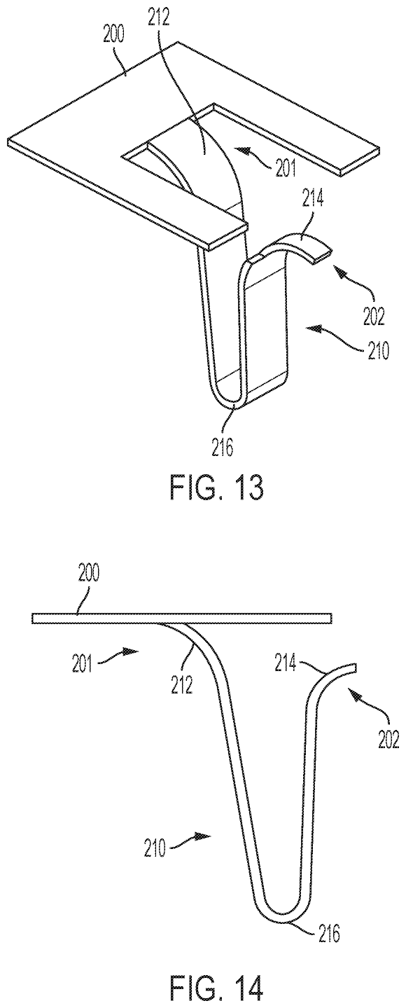

As best seen in FIGS. 13 and 14, the cantilevered arm 210 begins at a first curved portion 212 at its first end 201 and truncates at a second curved portion 214 at its second end 202. The arm also includes a U-shaped third curved portion 216 that connects the first curved portion 212 to the second curved portion 214. The second curved portion 214 is configured to contact with a conductive pad, and the third curved portion 216 is configured to contact with a sensed component such as the rotating tubular flange 38 shown in FIGS. 8 and 9.

The switch also includes a base 200 that is connected to the cantilevered arm 210. The base 200 is connected to the PCB to connect cantilevered arm to the PCB. The base and the arm together may form a single monolithic component.

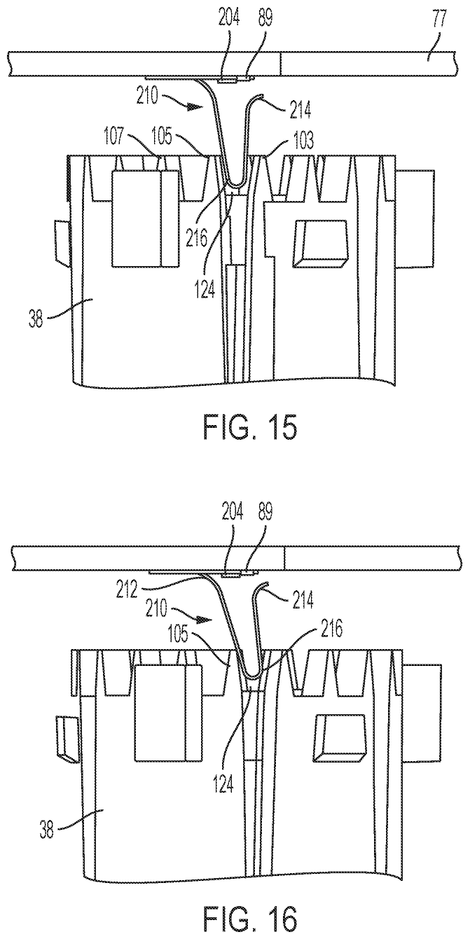

FIGS. 15-19 depict the cantilevered arm 210 of the switch interacting with the rotating flange 38 from FIGS. 8 and 9. FIG. 15 shows the arm 210 in an unstressed state, as the third curved portion 216 is situated within a recess 124 between two adjacent teeth 103, 105. The switch may be positioned in this state when the flange 38 is at its home or zero dose position, e.g. prior to use of the device, prior to setting a dosage, or after a dispense has completed and the device is ready for a dosage to be set.

In FIG. 16, the flange 38 has begun to rotate relative to the switch and the PCB 77. As a result, tooth 105 slides and pushes against the third curved portion 216 of the arm 210, causing the arm 210 to begin to deflect toward a direction out of the recess 124. The first curved portion 212 begins to move toward a straightened configuration, and the second curved portion 214 begins to move toward the conductive pad 89.

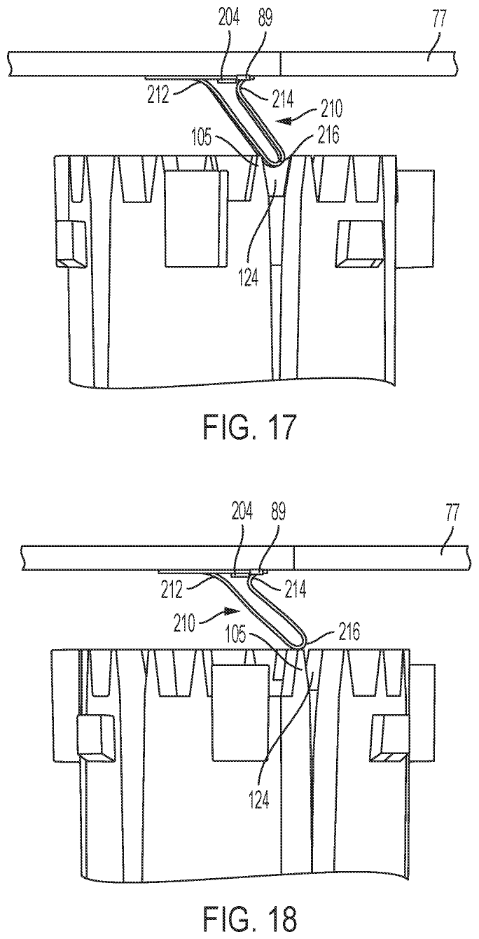

In FIG. 17, the flange 38 has rotated further than in FIG. 16, causing the tooth 105 to slide against and push the third curved portion 216 nearly completely out of the recess 124. The first curved portion 212 has moved even more toward a straightened configuration. As a result, the second curved portion 214 has made contact with the conductive pad 89, thereby closing the switch. The second curved portion also pressed against a blocking protrusion 204, which prevents the second curved portion from moving further toward the first curved portion 212, and may help to prevent the second curved portion from bouncing repeatedly against the conductive pad 89 in a rapid manner that may give rise to a noisy output signal.

In FIG. 18, the flange 38 has rotated further than in FIG. 17, and the third curved portion 216 has exited the recess 124 and is sliding across the top of tooth 105. The second curved portion 214 remains in contact with both the conductive pad 89 and the blocking protrusion 204. Blocking protrusion 204 has prevented the second curved portion 214 from moving closer to the first curved portion 212.

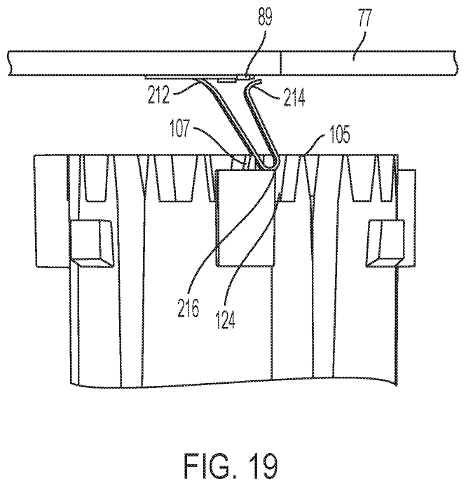

Finally, in FIG. 19, the flange 38 has rotated further than in FIG. 18, and the third curved portion 216 has stopped contacting tooth 105 and has now begun contacting the next adjacent tooth, 107. During this transition as the next tooth 107 is just beginning to push upon the arm 210, the arm, which is spring biased toward the position shown in FIG. 15, has swung back toward its unstressed state, thus causing the first curved portion 212 to move toward a more curved shape, resulting in movement of the third curved portion 216 toward a direction opposite to the rotation direction of the flange 38 and resulting in movement of the second curved portion 214 away from the conductive pad 84, thereby opening the switch. As the flange 38 rotates further, the cycle continues and the arm moves back toward the conductive pad to close the switch, and so on.

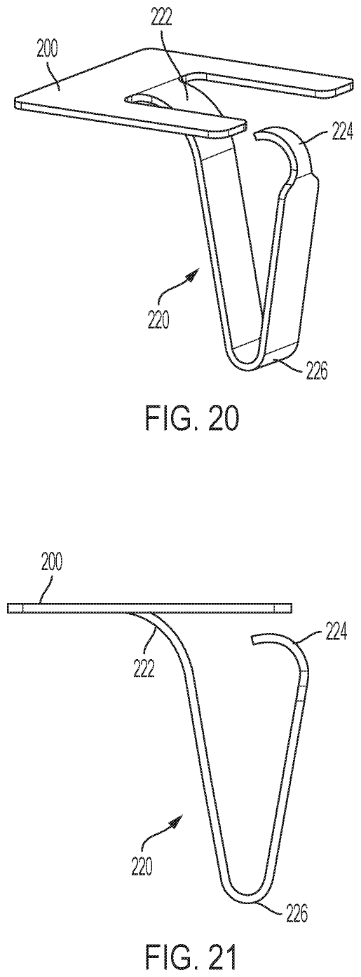

A first alternative embodiment of a switch is shown in FIGS. 20 and 21. This embodiment is similar to the embodiment shown in FIGS. 12-14, except that the second curved portion 224 is curved in the opposite direction. In the embodiment of FIGS. 12-14, the second curved portion 214 is curved such that, when the cantilevered arm is in an unstressed state, a curvature of the second curved portion faces away from the first curved portion 212. In the embodiment of FIGS. 20-21, the second curved portion 224 is curved such that, when the cantilevered arm is in an unstressed state, a curvature of the second curved portion faces toward the first curved portion 222. Otherwise, the base 200, first curved portion 222, and third curved portion 226 of the embodiment of FIGS. 20-21 are the same as those of the embodiment of FIGS. 12-14.

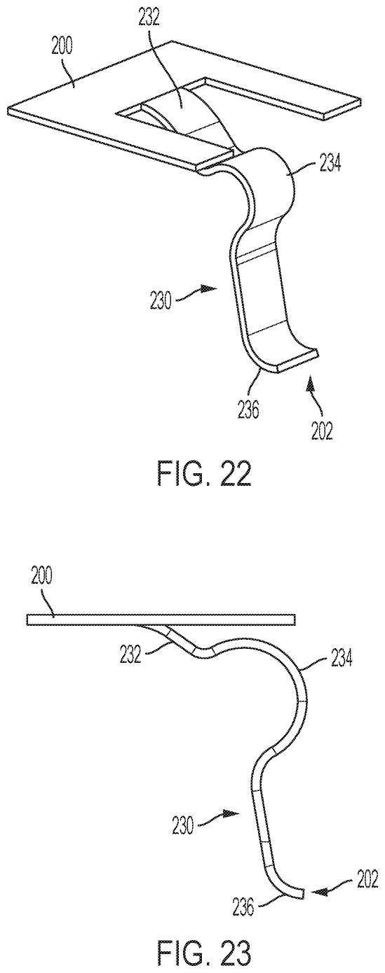

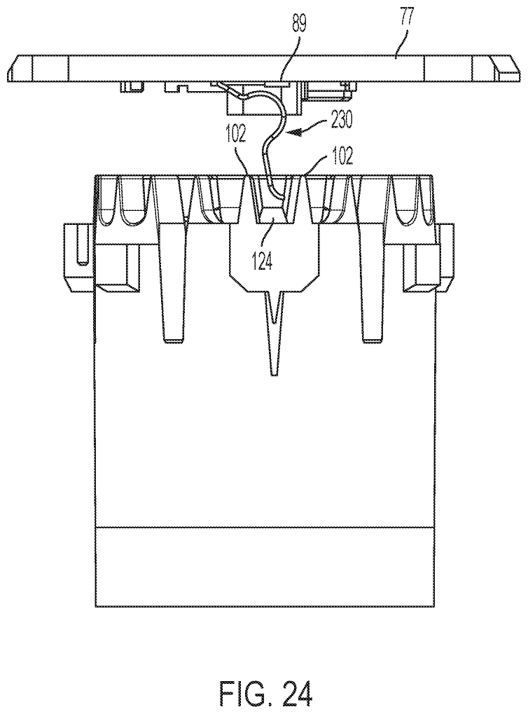

A second alternative embodiment of a switch is shown in FIGS. 22-23. In this embodiment, the first curved portion 232 is similar to the first curved portions of the other two embodiments, but the second the third curved portions are different. In this embodiment, the cantilevered arm 230 truncates at the second end 202 with the third curved portion 236 instead of the second curved portion 234. The third curved portion 236 is still configured to contact and slide against a sensed component such as the tubular flange, and the second curved portion 234 is still configured to contact with a conductive pad to close the switch. As a result, in this embodiment, the second curved portion 234 connects the first curved portion 232 to the third curved portion 236. Furthermore, the second curved portion 234 is U-shaped. To give this differently-shaped arm context, FIG. 24 shows the arm 230 interacting with teeth 102 of a rotating tubular flange 38, and interacting with a conductive pad 89.

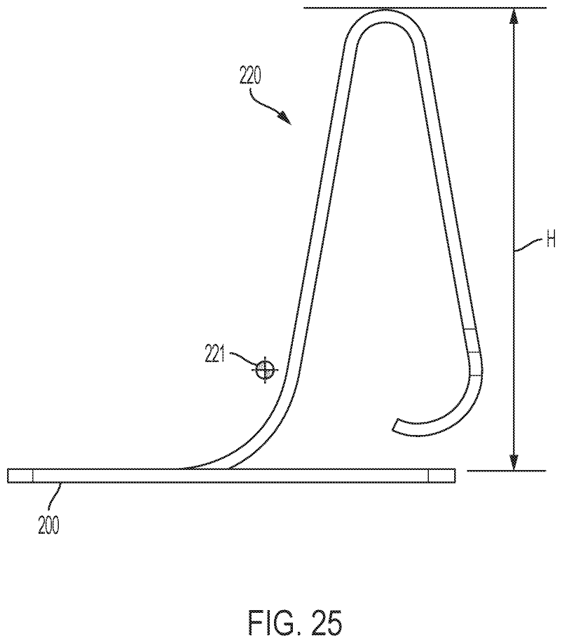

According to one aspect, the cantilevered arm may be shaped such that the center of mass of the arm is positioned over the base that is connected to the arm. For example, as shown in FIG. 25, in which the base and arm construct has been flipped upside-down, the center of mass 221 of the arm 220 is located over the base 200. Such a feature may aid in assembly of the base and arm with the PCB. For example, in some circumstances, it may be beneficial for a component to be able to stand up on its own, e.g. to utilize reflow soldering. Having the center of mass 221 of the arm 220 located over the base 200 permits the arm and base construct to stand up on its own on the PCB. It may also be beneficial for the center of mass to be relatively low, e.g. closer to the base than to the furthest point of the arm at height H. The embodiments of FIGS. 12-14 and FIGS. 22-23 are also examples of an arm shape having a center of mass that is positioned over the base.

According to one aspect, the printed circuit board (e.g., the printed circuit board 77 in FIG. 6) can include processing circuitry and/or logic to count the number of times the sensor (e.g., the sensor 86) is activated or triggered during an injection to determine a dose size of the injection. As described herein, the relative rotational movement between a dose-setting assembly and an actuator of the medication delivery device can be sensed in order to determine the amount of a dose delivered by a medication delivery device, because the sensed relative rotational movements can be correlated to the amount of the dose delivered.

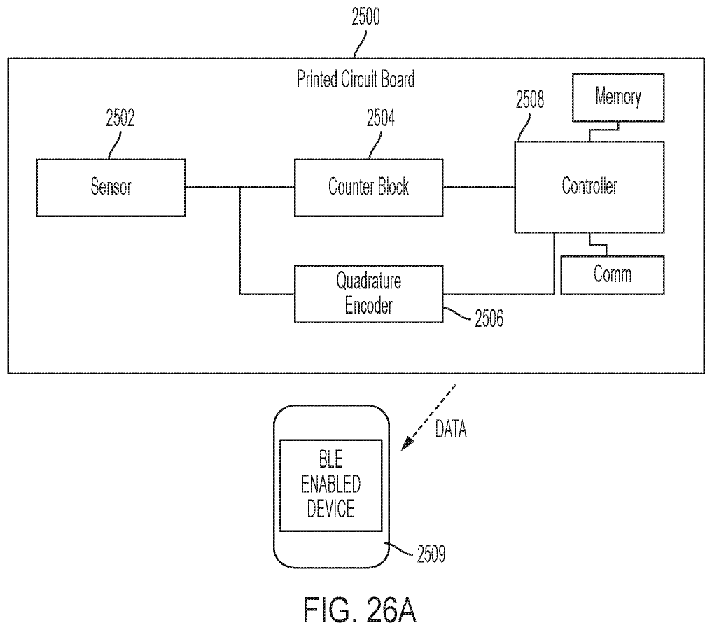

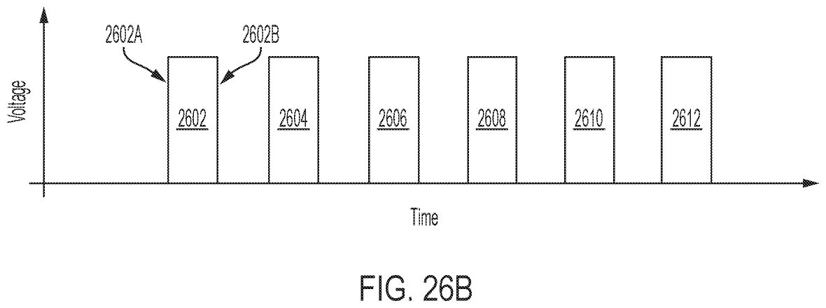

FIG. 26A is an exemplary block diagram pictorially illustrating functional aspects of a printed circuit board 2500 for processing signals from a sensor 2502, according to some embodiments. The sensor 2502 is in communication with both the counter block 2504 and the quadrature encoder 2506, which are both in communication with the controller 2508. The sensor 2502 can be, for example, the sensor 86 in FIG. 6. For example, as described herein the sensor 86 may be configured to track the movement of the contact surface 111 and associate the movement with an output signal that is sent to the counter block 2504 and the quadrature encoder 2506, which can count the number of times the contact surface 111 slides into and out of the recesses 124 defined between the teeth 102 of the flange 38, and provide the determined count to the controller 2508 (which can be used to determine the dose of an injection).

Controller 2508 includes at least one processor (e.g., microprocessor) that executes software and/or firmware stored in memory of the controller. The software/firmware code contains instructions that, when executed by processor, causes the controller to perform the functions of the control logic and steps described herein. The illustrative controller includes a processing core, a memory, a power source, and a communication module. These components may be mounted to and communicate via the printed circuit board 2500. Controller 2508 is operative to perform the operations described herein, including determining the number of units indicative of total angular movement of the dose setting member used to determine the amount of medication delivered from medication delivery device based on the information received from sensor 2502. Controller 2508 may store the detected amount of units or angular movement and/or medication separately or together in a generated dose record in memory. The generated dose record may include time/date stamp, dose delivered amount, battery charge status, error log messages, etc. Controller 2508 may also transmit the dose record data representative of detected amount of medication or units or angular movement via communication port to a paired remote computing device 2509, such as a user's computer or smartphone or a computing cloud. The information may be transmitted from communication port via a wired or wireless communication protocol, such as a Bluetooth low energy (BLE) (shown), WiFi, NFC, or cellular wireless communication protocol.

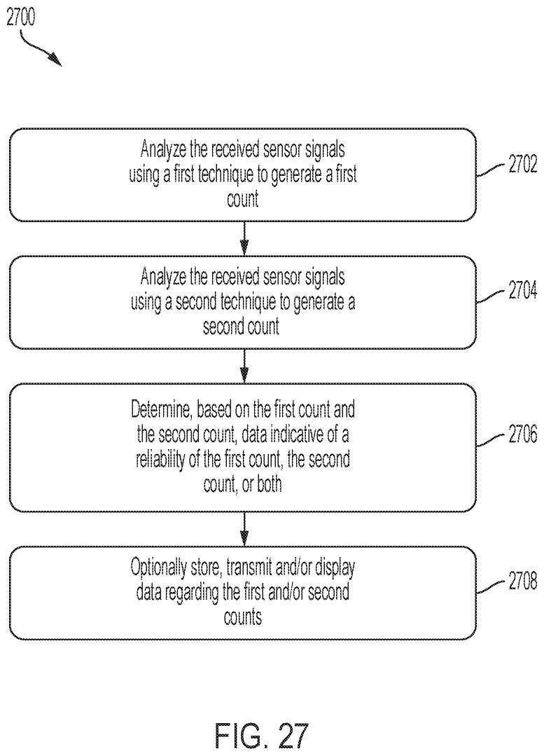

In some embodiments, the counter block 2504 and the quadrature encoder 2506 can be configured to process signals from the sensor 2502 using different techniques. Using different techniques can provide for some redundancy in measuring signals from the sensor 2502 (e.g., to gauge whether the measurements are accurate). FIG. 26B shows an exemplary set of six signals, six square waves 2602-2612 in this example, each of which can be generated by the sensor 2502 for a particular activation of the sensor 2502. The horizontal axis represents time, and the vertical axis represents voltage. FIG. 27 is a flow chart showing an exemplary computerized method 2700 for processing signals from a rotational sensor 2502, according to some embodiments. Referring to exemplary FIGS. 26B and 27, at step 2702, the counter block 2504 analyzes the received signals using a first technique to generate a first count of the signals. For example, the counter block 2504 can be configured to count the rising edges of each square wave 2602-2612, one of which is labelled for illustrative purposes as the rising edge 2602A of square wave 2602. At step 2704, the quadrature encoder 2506 analyzes the received signals using a second technique to generate a second count of the signals. For example, the quadrature encoder 2506 can be configured to count both the rising edges and falling edges of each square wave 2602-2612, one of the falling edges being labelled for illustrative purposes as the falling edge 2602B of the square wave 2602.

In some embodiments, the counter block 2504 and the quadrature encoder 2506 can be configured to process the signal from the sensor 2502 using different sampling rates. For example, the counter block 2504 can have a sample rate configured to sample the signal from the sensor 2502 to sense the rising edge of each signal, and the quadrature encoder 2506 can have a different sampling rate configured to sample the signal from the sensor 2502 to sense both the rising and falling edges of each signal. As an illustrative example, the counter block 2504 can be configured to use a 10 ms (100 Hz) sampling rate, while the quadrature encoder 2506 can be configured to use a 1 ms (1000 Hz) sampling rate.

At step 2706, the controller determines, based on the first count from the counter block 2504 and the second count from the quadrature encoder 2506, data indicative of a reliability of the first count, the second count, or both. For example, the controller 2508 can compare the counts to determine whether the counts match up, to gauge the accuracy of one count based on the other count, and/or the like.

In some embodiments, one of the counter block 2504 or the quadrature encoder 2506 can be a primary counting mechanism used to determine the actual count of the number of activations of the sensor 2502, and the other can be a secondary counting mechanism used to determine a measure of the reliability of the signal from the sensor 2502 and/or the count of the primary counting mechanism. For example, the counter block 2504 can be used to determine the actual number of times the sensor was activated (e.g., by counting the number of square waves in the signal from the sensor 2502, as described herein). The quadrature encoder 2506 can be used to analyze the same signal to determine whether the signal is a clean signal that should result in an accurate count by the counter block 2504, and/or whether the signal is too distorted/noisy to be able to result in an accurate count. For example, referring to the exemplary signal shown in FIG. 26B, if the quadrature encoder 2506 determined twelve transitions (e.g., six rising edges and six falling edges), then the controller 2508 can determine that a count of six by the counter block 2504 is likely an accurate count.

In some embodiments, the microcontroller 2508 can use a threshold when comparing the count determined by the counter block 2504 to the count from the quadrature encoder 2506 to determine whether the count of the counter block 2504 is reliable. For example, if the signal from the sensor 2502 includes some noise, the counter block 2504 may still achieve an accurate count when using a lower sampling rate than the quadrature encoder 2506 (e.g., because the lower sampling rate could filter out some of the noise in the signal). For example, the controller 2508 may determine that the count from the counter block 2504 is usable if it is within approximately 2-3 times the count determined by the quadrature encoder 2506. Assuming that the sensor generates six activation signals (e.g., six square waves) and that there is some noise, such that the quadrature encoder 2506 detects more transitions than the six leading and six trailing edges, if the counter block 2504 determines a count of six and the quadrature encoder 2506 determines a count of between 12-20 transitions, then the controller 2508 can register a dose of six units. However, if the quadrature encoder 2506 determines a count greater than 20, then the controller 2508 can consider the signal from the sensor 2502 to be too noisy (e.g., and doesn't register the count as a dose).

At step 2708, the medication delivery device can optionally store, transmit and/or display the data regarding the first and/or second counts. In some embodiments, the medication delivery device can include a display data regarding the first and/or second counts on a display device of the display (e.g., on a user interface, using one or more lights, using blinking lights and/or colored lights, and/or the like). In some embodiments, the medication delivery device can transmit the data regarding the first and/or second counts to a remote computing device. As described herein, the medication delivery device can be configured to communicate with a remote computing device. For example, the medication delivery device can be configured to communicate with a smartphone running an application. If the controller 2508 determines, based on the counts from the counter block 2504 and the quadrature encoder 2506, that the count generated by the counter block 2504 is reliable (e.g., and therefore can determine an estimate of the injected dose), then the controller 2508 can cause the medication delivery device to send to the remote computing device the computed dose size. Alternatively, if the controller 2508 determines that the count is not reliable, then the medication delivery device can be configured to not transmit data to the remote computing device and/or to transmit data indicative of an unknown dose injection or a potential dose injection event. An application running on the remote computing device can be configured to prompt the user to enter and/or verify an injection amount. For example, even for a count deemed accurate, the remote computing device may prompt a user to verify the injection amount. As another example, for a count deemed potentially inaccurate, the computing device can prompt the user to enter an injection amount.

In some embodiments, as described herein a user can press the dose button (e.g., dose button 30) at a desired pressure that, in turn, controls the speed of the injection. Therefore, in some embodiments the flange 38 can rotate at different speeds for each injection event. As an illustrative example, depending on the pressure applied to the dose button, the flange 38 can rotate from as slow as 1 Hz to as fast as 200 Hz.

In some embodiments, the controller 2508 can be configured to use the quadrature signal from the quadrature encoder 2506 to measure the speed of the injection. For example, if the quadrature encoder 2506 can be configured to sense both the rising and falling edges of each signal from the sensor 2502, the controller 2508 can determine the time between each sensor 2502 activation, which can be used to determine the overall speed of the injection.

In some embodiments, as discussed further herein, the controller 2508 can measure the time of each injection, such as the start time of the injection, the end time of the injection, the duration of the injection, and/or the like. The medication delivery device can be configured to store each injection event as a dose record. The medication delivery device can transmit dose record data to a remote computing device and/or the cloud, as described further herein.

In some embodiments, one signal (e.g., one pin of the controller 2508) can wake up the controller 2508 and cause the counter block 2504 and the quadrature encoder 2506 to sense signals from the sensor 2502. For example, activating the dose button can transmit a signal that can be used to activate each of the controller 2508, the counter block 2504, and the quadrature encoder 2506.

It should be appreciated that FIG. 26A is intended to illustrate functional aspects of the printed circuit board, and therefore the actual circuitry, logic and/or software can be implemented in various ways without departing from the spirit of the techniques described herein. For example, the counter block 2504 and/or the quadrature encoder 2506 can include dedicated hardware configured to perform the associated functionality described herein. As another example, the counter block 2504 and/or the quadrature encoder 2560 can be implemented as part of the hardware of the controller 2508 (e.g., a microcontroller). As a further example, the counter block 2504 and/or the quadrature encoder 2506 can be implemented as part of the sensor 2502. As another example, since both the counter block 2504 and the quadrature encoder 2506 can be used to process signals from the sensor 2502 to determine a dose size of an injected dose, some embodiments can include only one of the counter block 2504 or the quadrature encoder 2506.

According to one aspect, a user may receive digital feedback regarding how much medication remains within the medication delivery device. When the medication level of the medication delivery device becomes low, the user may receive an alert from the medication delivery device and/or from an external device informing the user of the low medication level. For example, the user may receive the alert from a mobile device, e.g. from an app, text message or SMS. An alert may be visual, auditory, tactile (e.g. a vibration) or any combination thereof.

As described herein, the printed circuit board can include non-volatile memory. The non-volatile memory can be used to store various usage information about the medication delivery device to store a history of the medication delivery device (e.g., manufacturing data, when the device was first activated, how many days the device was used, the number of injections, the dose of each injection, the time of each injection, etc.). Since the memory is non-volatile, the medication delivery device can be saved and/or sent back to the manufacturer for analysis.

In some embodiments, the medication delivery device can include one or more aspects that control at what point the medication delivery device stores and/or discards data that would be written to the non-volatile memory (e.g., if the non-volatile memory is a permanent write-only memory). For example, the medication delivery device can be configured to include a field or flag that the controller checks prior to writing data, such that the flag can be used to control whether the controller writes data to the non-volatile memory or discards the data without writing it to the non-volatile memory. The flag can be set to a first value during the supply chain and/or during manufacturing to disable writing to the non-volatile memory. For example, if inadvertent movement during manufacturing causes the sensor to output signals (e.g., which could be interpreted as dose injection information), having the flag set to a value representing a disabled state can prevent the microcontroller from writing data to the non-volatile memory. Upon the occurrence of an appropriate trigger (e.g., a user connecting a pen to an application running on a remote computing device, depressing the dose button, etc.), the microcontroller can be configured to change the flag to a value representing an enabled state so that when the user operates the medication delivery device the microcontroller writes data to the non-volatile memory. Once the flag is updated, the microcontroller will store data, such as dose injection information, to the non-volatile memory.

In some embodiments, the medication delivery device can include one or more aspects that can be used to control whether a medication delivery device is in an activated state or in a hibernation state (e.g., to control the power consumption of the medication delivery device to conserve battery usage). During manufacturing and/or the supply chain of the medication delivery device, the device can be initially configured in a hibernation state such that the controller is in a low-power state to avoid consuming battery life. The controller can include a timer that, once started upon activation of the medication delivery device, if no activity is detected by the medication delivery device before the timer ends (e.g., no data indicative of an injection, connection to an application running on a remote device, etc.), the controller places the medication delivery device back into a hibernation state. In some embodiments, the timer duration can be a number of hours, a number of days, a number of weeks, and/or any other predetermined time period. In some embodiments, the timer can be implemented using a counter, and the timer duration can be a number of clock pulses (e.g., representative of a desired number of hours, days, etc.). Switching the medication delivery device back into a hibernation state can be implemented such that it is not perceived by a user; the medication delivery device can be activated and function normally when a user activates the medication delivery device, even if the medication delivery device was previously placed back into a hibernation state.

In some embodiments, the timer can be implemented in conjunction with the flag used to prevent writing data to the non-volatile memory. For example, if the controller is activated and taken out of a hibernation state, the controller can check the value of the flag. If the controller determines that the flag is set to disable writing to the non-volatile memory, the controller can place the medication delivery device back into a hibernation state. Alternatively, if the controller determines that the flag is set to enable writing to the non-volatile memory, the controller can proceed with entering the activated state.

In some embodiments, the techniques can be used to manage multiple medication delivery devices. For example, a user may have access to and/or may be using multiple medication delivery devices. Each of the medication delivery devices can include unique identification information so that each device can be uniquely identified. As described herein, the medication delivery devices can communicate with an application running on a remote computing device. Each time a user starts using a new medication delivery device, the application can create an entry for the device and keep track of injection information and other information associated with the medication delivery device. For example, for some medications the medication delivery devices can be kept in cold storage prior to use, and are recommended to be used within a predetermined period once first used and removed from cold storage (e.g., within 28 days). Therefore, once first used, the application can keep track of the date and time each medication delivery device is activated in order to provide the user with information regarding when the user should use each medication delivery device.

In some embodiments, the user may be advised to refill their medication and/or, for re-useable medication delivery device, replace the medication cartridge with a new cartridge. In some embodiments, a refill request may be automatically sent to a pharmacy when a medication level is detected to be low.

In some embodiments, the application (e.g., in conjunction with dose information determined by the medication delivery device and/or input using the application) can keep track of how much medication is left in each medication delivery device. The application can manage injection delivery devices to try and use all of the existing doses prior to expiration. For example, the application can include a visual indication of how much medication is left in the medication delivery device and/or when the medication delivery device will expire. In some embodiments, if a medication delivery device only has a certain number of units left, the application can instruct the user to use the medication delivery device so that the user can use the available doses prior to expiration.

The method by which medication level is determined may be accomplished in different ways. In some embodiments, an external device stores a record of the level of medication remaining in a medication delivery device. The medication delivery device may send communications to the external device, informing the external device of the dosage that was delivered from each medication delivery event. The medication delivery device may determine such dosage information from the sensor described above. The external device may then calculate and store the amount of medication remaining after each medication delivery event. For example, the external device may subtract the delivered dosage amount from the last-known remaining amount of medication.

In some embodiments, the information regarding the amount of medication remaining in a medication delivery device may be calculated and/or stored by the medication delivery device itself. The medication delivery device may then communicate to an external device how much medication remains in the medication delivery device.

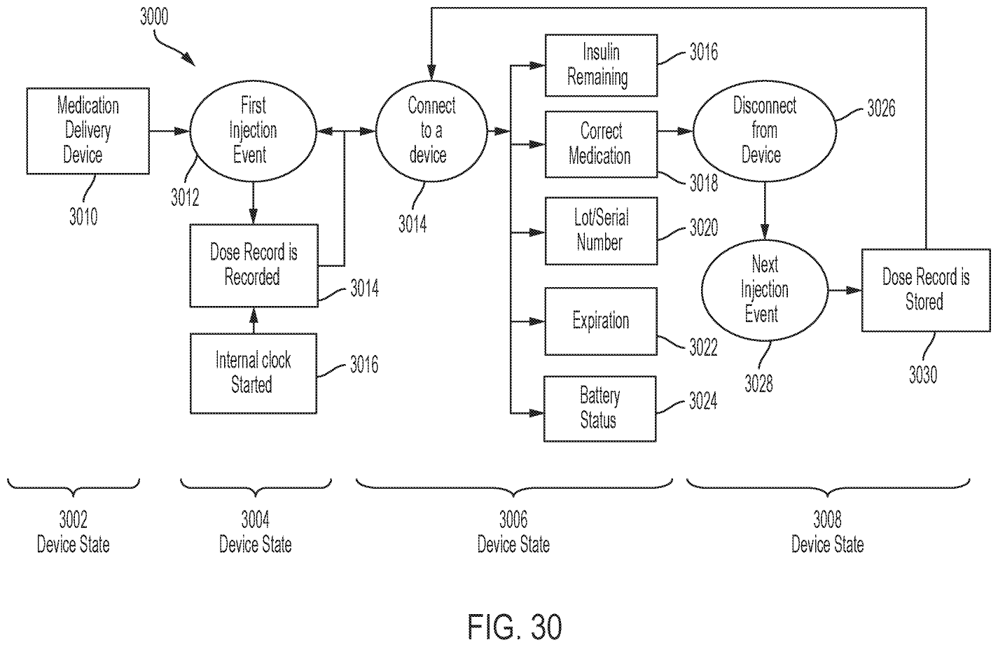

As described herein, the medication delivery device can operate in various device states and can store various information that can be transmitted to remote computing devices. FIG. 30 is a diagram 3000 illustrating various states and associated functionality of a medication delivery device, according to some embodiments. At device state 3002, the medication delivery device 3010 is in a hibernation state (e.g., a low power mode, with no wireless communication). At device state 3004, the medication delivery device 3010 is in an active state, during which the medication delivery device 3010 is available for communication with remote computing devices. During the active state 3004, the medication delivery device performs operations as discussed herein, including activating the sensor upon detection of a first injection event 3012, determining and recording a dose record 3014, and starting the device's internal clock at 3016.

At device state 3006, the medication delivery device 3010 is in an active state during which the medication delivery device 3010 is communicating with a remote computing device (not shown, e.g., an application running on a smartphone). The medication delivery device 3010 connects (3014) to a remote computing device, and the medication delivery device 3010 can transmit various data to the remote computing device (e.g., upon request by the remote computing device). As shown as examples in FIG. 30, the medication delivery device 3010 can transmit data indicative of one or more of an amount of insulin remaining 3016, an indication of whether the medication in the medication delivery device 3010 is a correct medication 3018, manufacturing and/or supply chain information for the medication delivery device 3010, such as the lot and/or serial number 3020, an expiration of the medication 3022, and/or a battery status 3024.

At device state 3008, the medication delivery device disconnects from the remote computing device (3026) and is active and available for communication with another remote computing device (the same and/or a different device). The medication delivery device 3010 detects a next injection event at 3028, stores the dose record for the injection event at 3030, and proceeds back to 3014 to connect to a device and transmit data requested by the device. The steps 3014 through 3030 can repeat as necessary to process the doses administered by the medication delivery device 3010.

As described herein and in conjunction with FIG. 30, the various manufacturing and medication data is stored on the medication delivery device, and can therefore be provided to remote computing devices. In some embodiments, the medication delivery device can determine aspects of the data sent to a remote computing device, such as determining the battery status based on battery voltage information. In some embodiments, the medication delivery device can provide the remote computing device with data (e.g., voltage information) that the remote computing device can use to determine the battery status. For example, the remote computing device can be configured to use one or more voltage curves to estimate the battery life left for the medication delivery device. In some embodiments, since various types and/or manufacturers of batteries can be used, different voltage curves may be used to estimate the remaining battery life. The remote computing device can use manufacturing data for the medication delivery device (e.g., the lot/serial number) to look-up data regarding the battery of the medication delivery device in order to determine an appropriate voltage curve to use to estimate the battery.

As another example, in some embodiments the medication delivery device and/or the remote computing device can use data from the medication delivery device to determine data indicative of an effectiveness of an administered dose. For example, the medication delivery device can store data regarding the temperature of the medication delivery device (e.g., at a time of injection, over the life of the medication delivery device, and/or the like), which can be used to determine an efficacy of the dose since temperature can impact the efficacy of the medication in the medication delivery device.

In some embodiments, tracking the amount of medication remaining in a medication delivery device may be used for patient adherence purposes. A user, caretaker, healthcare provider, insurance payer, and/or a company creating the medication may wish to monitor whether the user is taking the medication at the prescribed amounts and/or times. In some embodiments, such information may be used in conjunction with other devices to improve treatment for the patient. For example, the medication delivery device may be used in conjunction with a glucose meter. Dosages delivered to a patient may be paired with glucose level information to determine information such as efficacy of the medication, efficacy of the patient's regimen, etc. Such information may help to improve patient treatment, e.g. by suggesting possible ways to improve the patient's regimen.

According to one aspect, a medication delivery device may have the ability to assist a user with finding the location of the medication delivery device. The inventors have appreciated that a user may, at times, have trouble finding their medication delivery device, particularly if it is portable and can be used in different locations. The inventors have recognized the need for a device location assist feature to help the user locate the device.

In some embodiments, the location of a medication delivery device is tracked by one or more mobile devices. For example, a medication delivery device may be configured to communicate with one or more mobile devices or other external devices such as a remote server. The communication may be one-way communication or two-way communication.

In some embodiments, the medication delivery device periodically advertises information such as a unique identifier. A mobile device may periodically scan for medication delivery devices, and if the advertising medication delivery device is in communication range with the mobile device, the mobile device would receive the communication from the medication delivery device. The mobile device, which may have a built-in GPS or other location-identifying ability, may then associate a location with each received communication. Particularly if the communication protocol between the medication delivery device and the mobile device is a short distance communication protocol, such as Bluetooth, the mobile device may assign the mobile device's own present location, or a radius around the mobile device's own present location, as the location of the medication delivery device. In some embodiments, when the mobile device no longer receives communications from the medication delivery device, indicating that perhaps the medication delivery device has been moved out of communication range from the mobile device, the mobile device stores a last-known location of the medication delivery device, which may be when the mobile device last received communication from the medication delivery device. This last-known location may be presented to a user to help the user determine the location of the medication delivery device.

In some embodiments, when a mobile device is brought into communication range with a medication delivery device, the mobile device may alert a user that a medication delivery device is nearby. This may be used to help the user physically find the medication delivery device.

In some embodiments, when a mobile device senses that a medication delivery device is no longer in communication range with the mobile device (e.g. the mobile device does not receive an advertisement from the medication delivery device within an expected time period), the mobile device may alert the user the medication delivery device is no longer nearby, or at least no longer close to the mobile device itself. Such a feature may help the user to avoid forgetting to bring the medication delivery device when the user leaves a location.

In some embodiments, multiple mobile devices may cooperate to help locate a medication delivery device. For example, a group of mobile devices may be configured to scan periodically for medication delivery devices. When one of the mobile devices locates a medication delivery device (e.g. by sensing that the medication delivery device is in communication range), the mobile device may communicate the identity and/or location of the found medication delivery device to the rest of the mobile devices. This may be used, for example, in a household setting where members of the household each have their own mobile device.

In some embodiments, the medication delivery device may include a built-in speaker. To help a user find the medication delivery device, the speaker may be triggered by the user to emit a sound. In some embodiments, a user may use a mobile device to trigger the speaker to emit a sound.

In some embodiments, the medication delivery device itself may have a built-in GPS or other location-identifying ability. The medication delivery device may communicate its location to a mobile device or other external device, such as directly to a remote server.

According to one aspect, the date, and in some embodiments, time, at which a medication delivery device is used for the first time is tracked.

One example use case for such a feature is determining medication expiration. For example, in some embodiments, a medication delivery device may communicate to an external device that user has opened, turned on, or otherwise activated the medication delivery device for the first time. The external device may check whether the medication has expired, by, for example, looking up an identification number of the medication delivery device in an expiration date database.

Another example use case for such a feature is to assist in supply chain management. Referring further to FIG. 30, various manufacturing and supply chain data can be stored by the medication delivery device 3010, such as the manufacturing information (e.g., lot/serial number) 3020, expiration data 3022, and battery status data 3024. Additional information can be maintained, such as initial activation data, temperature data, dose records, and/or other data as described herein.

In some embodiments, the remote computing device can also interface with remote servers and/or cloud computing devices to process the manufacturing/supply chain data. For example, the remote computing device can use manufacturing data from the medication delivery device to determine additional information regarding the medication delivery device, such as when the medication delivery device was manufactured, a period between manufacture and first use, and/or the like. As another example, the remote computing device can obtain information determined by the medication delivery device throughout the manufacturing process, such as temperature through the supply chain, battery life throughout the supply chain, and/or the like. As a further example, the medication delivery device information can be used to identify and/or prevent use of counterfeit devices.

In some embodiments, injection data, such as the counts determined by the counter block 2504 and the counts from the quadrature encoder 2506, can be used for manufacturing purposes. For example, the injection data can be used to monitor the performance of the devices during manufacturing. In some examples, the count data can be used to understand the performance of products across different batches. For example, count data and/or other data can be used to identify a bad batch of devices (e.g., to flag the batch in the system and/or to notify users of the devices to discard those devices). Such data can be used for process improvement, such as to identify manufacturing problems, material problems, part problems, and/or the like.

The supply chain data can be used for various purposes by the medication delivery device and/or the remote computing device. For example, knowing when a specific medication delivery device has been activated for the first time may give a manufacturer important supply chain information, for example, how long it takes a medication delivery device to reach a user and be used by a user after the manufacturer has released it for sale. When communicating first use to an external device, the medication delivery device may also communicate its specific identification number to permit a manufacturer to associate the information to a known device and store the information in a database. The information can be categorized by device type, geography, etc.

In some embodiments, the time elapsed from first use of a medication delivery device may be monitored. With some types of medications and medication delivery devices, the medication in a medication delivery device expires after a certain amount of time has elapsed since the medication delivery device was first used to deliver an amount of the medication. This may apply in particular to multi-dose type medication delivery devices. As such, the medication delivery device may detect when the user has actuated the device to deliver medication for the first time. In some embodiments, the device may then begin an internal timer countdown and alert a user that the medication has expired when the timer reaches a predetermined time. Examples of an alert include turning on, off, or blinking a light and/or using a light of a certain color, an auditory sound, a vibration, or any combination thereof. In some embodiments, the medication delivery device may prevent the user from actuating the device, e.g. with a physical and/or electrical lockout that makes delivery impossible. In some embodiments, when the medication delivery device detects that the user has actuated the device to deliver medication for the first time, the medication delivery device may communicate to an external device that first time delivery has occurred. The external device may then begin a countdown to the expiration of the medication. When the countdown as completed, the external device may send an alert to the user and/or communicate to the medication delivery device that the medication has expired.

According to one aspect, a temperature of the medication may be monitored. Medication temperature may be monitored directly or indirectly. One example of direct measurement includes placing a temperature sensor in actual contact with the medication. One example of indirect measurement includes using a temperature sensor to measure the temperature of a region or component close to the medication to approximate what the actual temperature of the medication is. For example, in one embodiment, a temperature sensor is located at the PCB of a medication delivery device. Another example of indirect measurement includes directly measuring the temperature of a material within the medication delivery device that behaves similarly to the actual medication when exposed to various temperature environments.

Temperature measurements may occur periodically. Information relating to the measured temperature may be stored within the medication delivery device, may be communicated to an external device each time a measurement occurs or in batches, or any combination thereof.

When the medication delivery device sensors that the measured temperature is outside an acceptable temperature range, referred to herein as a “temperature excursion,” a variety of responses may occur. In some embodiments, the medication delivery device alerts the user directly and/or communicates the information to one or more external devices, which may in turn alert the user. The alert may occur in real-time when the temperature excursion is detected, or may occur the next time the user uses the medication delivery device. In some embodiments, when a temperature excursion is detected, the medication delivery device may store and/or communicate to an external device the time and/or date of the temperature excursion, as well as the measured temperature.

According to one aspect, a medication delivery device and/or an external program that communicates with the medication delivery device, such as a mobile device app, may include security features for controlling wireless communication between the medication delivery device and an external device. In some embodiments, the medication delivery device includes a bond management feature that prevents unwanted access to the medication delivery device from third parties. In this bond management feature, a user has already paired their medication delivery device to the user's mobile device, which may be running an app that is specialized for use with the medication delivery device. If a different mobile device tries to connect with the medication delivery device, the user may receive a notification that a third party is attempting to connect with the medication delivery device. The user may grant or deny permission for the third party to connect with the medication delivery device. In some embodiments, this setting may be “remembered” by the app and/or medication delivery device to avoid repeated notifications. In some embodiments, the app may be configured to include a menu that allows a user to change past authorization settings, e.g. to grant access to a previously denied third party mobile device, or to deny access to a previously approved third party mobile device.

Bluetooth Low Energy devices can be difficult to connect to remote computing devices like smartphones in a secure way, because most use a secure connection (which may take a long time to establish), then send plaintext data over the secure connection. In order to simplify the pairing process and make it hassle-free for users, a new pairing process is described below. In the new method of pairing, the Bluetooth connection is left open so that connection can happen fast, but the data sent over the open connection is encrypted using a symmetric key. It is believed by the inventors that this method of pairing can enable users to pair a BLE device to a smartphone in a quick fashion while still establishing a secure connection.

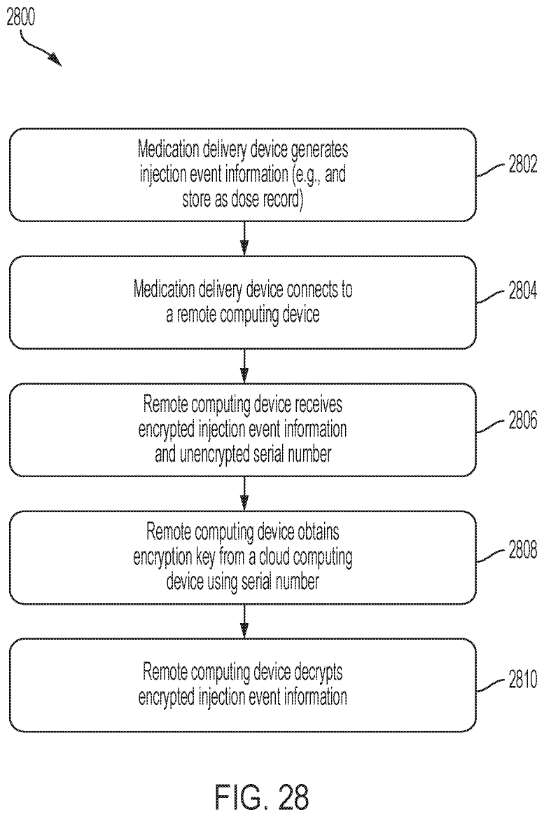

FIG. 28 is a flow chart showing an exemplary encryption scheme 2800 that uses open (unencrypted) communication channels to exchange encrypted information and accesses encryption information from a cloud computing device, according to some embodiments. At step 2802, a medication delivery device generates injection event information (e.g., in response to a user performing an injection event). The injection event information can be recorded in a dose record, as described herein. At step 2804, the medication delivery device connects to a remote computing device, such as by using open/unencrypted wireless communication channel(s). For example, for a Bluetooth communication, the medication delivery device and remote computing device can connect by agreeing to communicate at a specified time and frequency (e.g., channel), but without performing any handshake process and/or encryption key exchange.

At step 2806, the medication delivery device transmits, and the remote computing device receives, data for the injection event, which can include encrypted injection event information and unencrypted data used to obtain an encryption key to decrypt the encrypted data (e.g., in this example, a unique identifier of the medication delivery device, such as a serial number). In some embodiments, the medication delivery device can be configured such that the medication delivery device transmits data upon request by the remote computing device. Therefore, the remote computing device can request injection data from the medication delivery device, and the medication delivery device can transmit any available data (e.g., which has not yet been transmitted to the remote computing device). Since the encryption protocol can be managed separately from the wireless communication protocol, the devices can be configured to transmit data in any desired format, and which can be modified over time. For example, while the encryption key is requested using an identifier for the medication delivery device, that can be changed if desired. As another example, the formatting of the dose record data can be configured and/or changed as desired.

At step 2808, the remote computing device uses the unencrypted data to obtain a decryption key from a cloud computing device (e.g., a cloud computing device provided by the company that provided the medication delivery device). At step 2810, the remote computing device can decrypt the encrypted injection event information using the obtained decryption key.

In some embodiments, it can be desirable to avoid using provided encryption techniques. For example, it can be desirable to avoid needing to perform a handshake process required to set up encrypted communication channels (e.g., the handshake process used for encrypted Bluetooth communications). The handshake process can be burdensome to the user given the amount of time it can take, difficulties completing the handshake process, and/or the like. As another example, some devices store lists of paired devices, such that if a user uses a large number of medication delivery devices, which can result in a very long list of paired devices on the user's smartphone. Using a custom encryption technique in place of existing encryption techniques can avoid such problems and annoyances. Additionally, or alternatively, using a custom encryption technique can allow custom control of the encryption environment by the medication delivery device provider (e.g., allowing the provider to change the encryption protocol, encryption process, and/or the like).

As part of the encryption process, a medication delivery device user may be required to first register with the application provider before being able to use the application. For example, a user may need to create an account and perform various authentication requirements before being given access to the application. Additionally, the specific encryption keys used by a medication delivery device can be specific to the medication delivery device. Therefore, even if the application was hacked and the hacker was able to guess the identifying information of a medication delivery device, since the device can be located anywhere in the world the hacker would not be able to simply decrypt any medication delivery device communications.

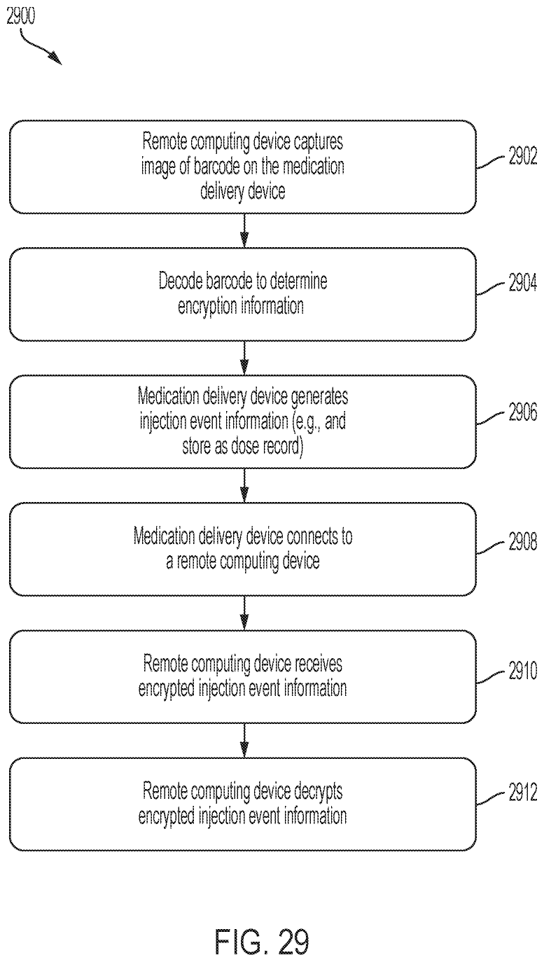

FIG. 29 is a flow chart showing an exemplary encryption scheme 2900 that uses open (unencrypted) communication channels to exchange encrypted information and determines encryption information from a barcode on the medication delivery device, according to some embodiments. At step 2902, the remote computing device captures an image of a barcode on the medication delivery device. The barcode can be, for example, a two-dimensional barcode disposed one the medication delivery device. At step 2904, the remote computing device decodes the barcode to determine encryption information (e.g., a private encryption key) associated with the medication delivery device. The barcode can include the encryption information as well as other information associated with the medication delivery device, such as a unique identifier/serial number for the medication delivery device. The application can add the medication delivery device (e.g., based on its serial number) to the list of devices being used by the user, and associate the encryption information with the device entry.

At step 2906, the medication delivery device generates injection event information (e.g., recorded in a dose record). At step 2908, the medication delivery device connects to a remote computing device, such as by using open/unencrypted wireless communication channel(s) as described herein. At step 2910, the medication delivery device transmits, and the remote computing device receives, encrypted injection event information. At step 2912, the remote computing device decrypts the encrypted injection event information received at step 2910 using the decryption key determined at step 2904.

In some embodiments, the medication delivery device can include a termination feature that disables wireless transmission when a user is finished using the medication delivery device. For example, if a user disposes of a medication delivery device, the termination feature can be used to prevent the barcode from being scanned and encrypted data from being read from the device and decrypted. In some embodiments, the termination feature can be activated after expiration of a predetermined time period, upon indication by the user in the application that the user is no longer using the medication delivery device, and/or the like.

In some embodiments, the medication delivery device can be configured to transmit wireless advertising packets for short range wireless communication protocols (e.g., which are used to connect to remote computing devices). The advertising packets can include various data, such as encryption information (e.g., an encryption key), an identifier for the medication delivery device, and/or the like. In some embodiments, since the communication protocol is only a short range protocol, the medication delivery device can transmit the advertising packets unencrypted. In such embodiments, the remote computing device therefore need not decrypt the advertising packets to obtain the medication delivery device serial number, etc. In some embodiments, the medication delivery device can encrypt the advertising packets. For example, a mobile application can obtain the information necessary to decode the advertising packets by decoding a barcode (e.g., as discussed in conjunction with FIG. 29). The mobile application can store a set of all active medication delivery devices and associated encryption information, and attempt to decode received advertising packets until the mobile application successfully decodes the received advertising packets.

According to one aspect, a user may be notified by an external device or by the medication delivery device itself if the medication delivery device is subject to a recall. In some embodiments, the medication delivery device broadcasts its unique identification number to an external device which may communicate with a remote server that has a database that associates recall information with the identification number. The medication delivery device may communicate directly with the remote server itself.

In some embodiments, an external device such as a mobile device alerts a user that the medication delivery device and/or the medication within the device is subject to a recall and should not be used. The alert may take different forms, including a message displayed by an app running on the mobile device, via text message, via SMS, via email, or any combination thereof.

In some embodiments, the medication delivery device may be instructed by the remote server and/or an intermediate external device such as a mobile device to display an alert informing the user that the medication delivery device and/or medication is subject to a recall and should not be used. In some embodiments, the medication delivery device may activate a physical and/or electrical lockout that prevents the device from being used.

According to one aspect, the medication delivery device may be used to detect failure to administer a dose in accordance with the user's prescribed regimen. For example, if a user accidentally administers two doses at once or too close in time, an external device or the medication delivery device itself will inform the user of the error. As other examples, a user may have accidentally or intuitionally skipped a dose, or may have used an incorrect dose.

Detection of these types of errors may give the user an opportunity to take remedial measures. The external device may provide suggestions to the user for remedial measures, may inform the user's healthcare provider, may connect the user to the healthcare provider, or any combination thereof.

In some embodiments, such errors are able to be monitored because the medication delivery device may be able to detect delivery of medication and may be able to detect the dosage that was delivered. The medication delivery device may communicate such information out to an external device.

Either the external delivery device or the medication delivery device itself may then determine whether such administrations were proper. In some embodiments, an external device or the medication delivery device itself may compare timings and dosages of actual administrations against expected administrations. If the actual administrations do not match with the expected administrations, then the external device and/or the medication delivery device may inform the user, e.g. that they have missed a dose, administered too much or too little of a dosage, or any combination thereof. In one illustrative embodiment, a medication delivery device communicates dosage amounts and delivery times to a mobile device. The mobile device then communicates with a remote server to determine whether this actual administration matches with an expected prescribed regimen. If the actual administration does not match with the expected regimen, then the mobile device alerts the user to an administration error. In situations with a missed dose, the remote server may communicate with the mobile device the inform the mobile device that a dosage should have been administered. If the mobile device has not received information from the medication delivery device indicating that the dosage was administered, the mobile device may then send an alert to the user reminding the user to take their medication.

In some embodiments, the medication delivery device can include an on-board oscillator-based clock (e.g., on the printed circuit board) that incrementally counts clock pulses over time. Oscillator-based clocks can be less-accurate than other types of clocks, such as quartz-based clocks, and can therefore be subject to drift that can cause the clock measurements to become inaccurate over time. For example, the clock may have little or no drift on the first day that the medication delivery device is activated, but the drift can increase each day. For example, after two days the drift may be four minutes off, at five days the drift may be ten minutes off, and so on.

In some embodiments, the medication delivery device can be configured to periodically synchronize the clock time with a remote clock source (e.g., by connecting to an application on a remote device). Such synchronization can reduce the amount of drift of the clock over time. For example, upon an initial activation of the medication delivery device, the clock value can be zero and the clock can start counting at that point. Periodically, the medication delivery device is reset (e.g., to zero and/or to a particular value), such as each predetermined time period (e.g., number of hours, days, etc.), each injection time, each time the medication delivery device connects to a remote device, and/or the like. As another example, the mobile application can keep track of a threshold counter value that is used to determine when to reset the clock (e.g., a reset can be caused when the clock counter is greater than the threshold). For example, the threshold can be set to a count value indicative of 12 hours, one day, two days, and/or the like.

In some embodiments, the clock resets can be controlled by a remote device. For example, if using a counter threshold, when the mobile device determines that the medication delivery device counter is greater than the threshold, the mobile device can resynchronize the medication delivery device clock. The mobile device can store an initial activation time when the medication delivery device clock is first activated. Then the remote device can keep track of each reset event. For example, the mobile application can keep track of the number of resets, the time of each reset, the clock value at each reset, and/or any other relevant information.

The mobile device can use the stored clock information, including the reset event data, to determine the time of each injection. For example, upon initial activation the clock counter can be at zero, and the mobile device can record an initial activation time of 1:00 PM on day zero (e.g., recorded using a universal time, such as Greenwich Mean Time), which the application can store locally on the remote device. If the mobile device then receives data indicative of an injection, the remote device can determine an associated injection time based on the dose record data sent by the medication delivery device, which can include clock information such as the current clock counter and the number of resets. Continuing with the example above of an initial activation time of 1:00 PM on day zero, assume the mobile device receives dose record data with a clock counter indicative of 60 minutes, and no resets. The mobile device can be configured to determine that after 60 minutes, the clock likely has about one minute of drift, and can therefore assign the time of 1:00 PM+59 seconds=1:59 PM on day zero.

In some embodiments, the techniques can include performing a clock reset (e.g., synchronizing with the phone as described herein) based on one or more other triggers in addition to and/or instead of a periodic synchronization. In some embodiments, a clock reset can be triggered based on one or more factors that may influence the accuracy of the clock. For example, a clock may be affected by the battery life, temperature, moisture, movement, and/or the like. As an example, since oscillator-based clocks may be sensitive to rapid temperature changes, the device can be configured to synchronize the clock if a temperature change over a period of time is detected that is above a threshold configured for a clock synchronization.

The shown device is a reusable pen-shaped medication injection device, generally designated, which is manually handled by a user to selectively set a dose and then to inject that set dose. Injection devices of this type are well known, and the description of device is merely illustrative as the sensing system can be adapted for use in variously configured medication delivery devices, including differently constructed pen-shaped medication injection devices, differently shaped injection devices, and infusion pump devices. The medication may be any of a type that may be delivered by such a medication delivery device. Device is intended to be illustrative and not limiting as the sensing system described further below may be used in other differently configured devices.

Techniques operating according to the principles described herein may be implemented in any suitable manner. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media (shown schematically as “memory” in FIG. 26A). Such a computer-readable medium may be implemented in any suitable manner. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

Further, some techniques described above comprise acts of storing information (e.g., data and/or instructions) in certain ways for use by these techniques. In some implementations of these techniques—such as implementations where the techniques are implemented as computer-executable instructions—the information may be encoded on a computer-readable storage media. Where specific structures are described herein as advantageous formats in which to store this information, these structures may be used to impart a physical organization of the information when encoded on the storage medium. These advantageous structures may then provide functionality to the storage medium by affecting operations of one or more processors interacting with the information; for example, by increasing the efficiency of computer operations performed by the processor(s).

In some, but not all, implementations in which the techniques may be embodied as computer-executable instructions, these instructions may be executed on one or more suitable computing device(s) operating in any suitable computer system, or one or more computing devices (or one or more processors of one or more computing devices) (shown schematically as controller 2508 in FIG. 26A) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

A computing device may comprise at least one processor, a network adapter, and computer-readable storage media. A computing device may be, for example, a desktop or laptop personal computer, a personal digital assistant (PDA), a smart mobile phone, a server, or any other suitable computing device. A network adapter may be any suitable hardware and/or software to enable the computing device to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media may be adapted to store data to be processed and/or instructions to be executed by processor. The processor enables processing of data and execution of instructions. The data and instructions may be stored on the computer-readable storage media.

A computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

Various aspects are described in this disclosure, which include, but are not limited to, the following aspects:

1. A medication delivery device comprising: a housing comprising a reservoir sized sufficiently to hold medication; a dose button being rotatable relative to the housing to select a dose size of the medication for an injection; a printed circuit board; a switch mounted to the printed circuit board; and a controller in communication with the switch, the controller being configured to: receive a set of signals from the switch; generate, based on the set of signals, a first count of the set of signals using a first counting technique; generate, based on the set of signals, a second count of the set of signals using a second counting technique; and determine, based on the first count and the second count, data indicative of a reliability of the first count, the second count, or both.

2. The medication delivery device of aspect 1, wherein the controller includes a counter block configured to analyze the set of signals using the first counting technique to generate the first count.

3. The medication delivery device of aspect 2, wherein the counter block is configured to generate the first count by counting a number of signals in the set of signals.

4. The medication delivery device of aspect 3, wherein: each signal in the set of signals comprises an associated square wave, wherein each associated square wave comprises a rising edge at a beginning of the square wave; the counter block is configured to sample the set of signals at a sampling rate configured to capture each rising edge of each square wave; and generating the first count comprises counting the number of rising edges.

5. The medication delivery device of any one of aspects 1-4, wherein the controller includes a quadrature encoder configured to analyze the set of signals using the second counting technique to generate the second count.

6. The medication delivery device of aspect 5, wherein: each signal in the set of signals comprises an associated square wave, wherein each square wave comprises a rising edge at a beginning of the square wave and a falling edge at an end of the square wave; the quadrature encoder is configured to sample the set of signals at a sampling rate configured to capture each rising edge and each falling edge of each square wave; and generating the second count comprises counting the number of rising edges and the number of falling edges.

7. The medication delivery device of any one of aspects 1-6: wherein the switch comprises a contact surface arm extending from the printed circuit board, said arm configured to move to close or open the switch; and; and the medication delivery device further comprising a rotatable element that is rotatable relative to the printed circuit board, the rotatable element having a series of protrusions that are spaced from one another, the rotatable element being positioned to permit the protrusions to slide against the contact surface arm to move the switch to one of a closed state or an open state as the rotatable element rotates..

8. The medication delivery device of any one of aspects 1-6: wherein the switch comprises a piezoelectric sensor, a magnetic sensor, an accelerometer, an optical sensor, an interrupter sensor, an optical encoder, or some combination thereof.

9. The medication delivery device of aspect 8, wherein the magnetic sensor is a Hall effect sensor.

10. The medication delivery device of aspect 8, wherein the optical sensor is a reflective sensor.

11. A computerized method being executed by a controller of a medication delivery device, wherein the controller is in communication with a switch mounted to a printed circuit board, the method comprising: receiving a set of signals from the switch; generating, based on the set of signals, a first count of the set of signals using a first counting technique; generating, based on the set of signals, a second count of the set of signals using a second counting technique; and determining, based on the first count and the second count, data indicative of a reliability of the first count, the second count, or both.

12. The computerized method of aspect 11, further comprising analyzing, using a counter block of the controller, the set of signals using the first counting technique to generate the first count.

13. The computerized method of aspect 12, further comprising generating, by the counter block, the first count by counting a number of signals in the set of signals.

14. The computerized method of aspect 13, wherein: each signal in the set of signals comprises an associated square wave, wherein each associated square wave comprises a rising edge at a beginning of the square wave; and the method further comprises: sampling, by the counter block, the set of signals at a sampling rate configured to capture each rising edge of each square wave; and generating the first count comprises counting the number of rising edges.

15. The computerized method of any one of aspects 11-14, further comprising analyzing, using a quadrature encoder of the controller, the set of signals using the second counting technique to generate the second count.

16. The computerized method of aspect 15, wherein: each signal in the set of signals comprises an associated square wave, wherein each square wave comprises a rising edge at a beginning of the square wave and a falling edge at an end of the square wave; and the method further comprises: sampling, by the quadrature encoder, the set of signals at a sampling rate configured to capture each rising edge and each falling edge of each square wave; and generating the second count comprises counting the number of rising edges and the number of falling edges.

17. A medication delivery device comprising: a housing comprising a reservoir sized sufficiently to hold medication; a dose button being rotatable relative to the housing to select a dose size of the medication for an injection; a wireless communication module; and a controller in communication with the wireless communication module configured to: generate injection event information for the medication delivery device; connect, using the wireless communication module, to a remote computing device to wirelessly communicate with the remote computing device over an unencrypted wireless communication channel; and transmit, using the wireless communication module, encrypted data to the remote computing device over the unencrypted wireless communication channel, wherein the encrypted data comprises data indicative of the injection event information.

18. The medication delivery device of aspect 17, further comprising a two-dimensional barcode that encodes encryption data that can be used by a computing device to decrypt the encrypted data.

19. The medication delivery device of aspect 18, wherein the encryption data comprises a private encryption key.

20. The medication delivery device of any one of aspects 17-19, wherein transmitting the encrypted data further comprises transmitting an unencrypted identifier for the medication delivery device.

21. The medication delivery device of any one of aspects 17-20, wherein the controller is further configured to wirelessly transmit an advertising packet to advertise the presence of the medication delivery device for connection.

22. The medication delivery device of aspect 21, wherein the controller is further configured to encrypt the advertising packet.

23. The medication delivery device of any one of aspects 17-22, wherein connecting to the remote computing device to wirelessly communicate with the remote computing device over an unencrypted wireless communication channel comprises connecting to the remote computing device using an unencrypted Bluetooth communication channel.

24. A computerized method being executed by a controller of a medication delivery device, wherein the controller is in communication with a wireless communication module, the method comprising: generating injection event information for the medication delivery device; connecting, using the wireless communication module, to a remote computing device to wirelessly communicate with the remote computing device over an unencrypted wireless communication channel; and transmitting, using the wireless communication module, encrypted data to the remote computing device over the unencrypted wireless communication channel wherein the encrypted data comprises data indicative of the injection event information.

25. The computerized method of aspect 24, wherein transmitting the encrypted data further comprises transmitting an unencrypted identifier for the medication delivery device.

26. The computerized method of any one of aspects 24-25, further comprising wirelessly transmitting an advertising packet to advertise the presence of the medication delivery device for connection.

27. The computerized method of aspect 26, further comprising encrypting the advertising packet.

28. The computerized method of any one of aspects 24-27, wherein connecting to the remote computing device to wirelessly communicate with the remote computing device over an unencrypted wireless communication channel comprises connecting to the remote computing device using an unencrypted Bluetooth communication channel.

29. A computing device comprising at least one processor in communication with a memory configured to store machine-readable instructions that cause the at least one processor to: connect to a wireless communication module of a medication delivery device to wirelessly communicate with the medication delivery device over an unencrypted wireless communication channel, wherein the medication delivery device comprises (a) a housing comprising a reservoir sized sufficiently to hold medication, (b) a dose button being rotatable relative to the housing to select a dose size of the medication for an injection, and (c) a controller in communication with the wireless communication module; and receive, from the medication delivery device using the wireless communication channel, encrypted data over the unencrypted wireless communication channel, wherein the encrypted data comprises injection event information generated by the medication delivery device.

30. The computerized method of aspect 29, further comprising decoding, from a two-dimensional barcode on the medication delivery device, encryption data that can be used to decrypt the encrypted data.

31. The computerized method of aspect 30, wherein the encryption data comprises a private encryption key.

32. The computerized method of any one of aspects 29-31, wherein receiving the encrypted data further comprises receiving an unencrypted identifier for the medication delivery device.

33. The computerized method of any one of aspects 29-32, further comprising wirelessly receiving an advertising packet to advertise the presence of the medication delivery device for connection.

34. The computerized method of aspect 33, wherein the advertising packet is encrypted, and the method further comprises decrypting the advertising packet.

35. The computerized method of any one of aspects 29-34, wherein connecting to the medication delivery device to wirelessly communicate with the medication delivery device over the unencrypted wireless communication channel comprises connecting to the medication delivery device using an unencrypted Bluetooth communication channel.

36. A medication delivery device in any of aspects 1-35, comprising a medication.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A medication delivery device comprising:
a housing comprising a reservoir sized sufficiently to hold medication;
a dose button being rotatable relative to the housing to select a dose size of the medication for an injection;
a wireless communication module; and
a controller in communication with the wireless communication module configured to:
generate injection event information for the medication delivery device;
connect, using the wireless communication module, to a remote computing device to wirelessly communicate with the remote computing device over an unencrypted wireless communication channel; and
transmit, using the wireless communication module, encrypted data to the remote computing device over the unencrypted wireless communication channel, wherein the encrypted data comprises data indicative of the injection event information.

18. The medication delivery device of claim 17, further comprising a two-dimensional barcode that encodes encryption data that can be used by a computing device to decrypt the encrypted data.

19. The medication delivery device of claim 18, wherein the encryption data comprises a private encryption key.

20. The medication delivery device of claim 17, wherein transmitting the encrypted data further comprises transmitting an unencrypted identifier for the medication delivery device.

21. The medication delivery device of claim 17, wherein the controller is further configured to wirelessly transmit an advertising packet to advertise the presence of the medication delivery device for connection.

22. The medication delivery device of claim 21, wherein the controller is further configured to encrypt the advertising packet.

23. The medication delivery device of claim 17, wherein connecting to the remote computing device to wirelessly communicate with the remote computing device over the unencrypted wireless communication channel comprises connecting to the remote computing device using an unencrypted Bluetooth communication channel.

24. A computerized method being executed for execution by a controller of a medication delivery device, wherein the controller is in communication with a wireless communication module, the method comprising:
generating injection event information for the medication delivery device;
connecting, using the wireless communication module, to a remote computing device to wirelessly communicate with the remote computing device over an unencrypted wireless communication channel; and
transmitting, using the wireless communication module, encrypted data to the remote computing device over the unencrypted wireless communication channel wherein the encrypted data comprises data indicative of the injection event information.

25. The computerized method of claim 24, wherein transmitting the encrypted data further comprises transmitting an unencrypted identifier for the medication delivery device.

26. The computerized method of claim 24, further comprising wirelessly transmitting an advertising packet to advertise the presence of the medication delivery device for connection.

27. The computerized method of claim 26, further comprising encrypting the advertising packet.

28. The computerized method of claim 24, wherein connecting to the remote computing device to wirelessly communicate with the remote computing device over the unencrypted wireless communication channel comprises connecting to the remote computing device using an unencrypted Bluetooth communication channel.

29. A computing device comprising at least one processor in communication with a memory configured to store machine-readable instructions that cause the at least one processor to:
connect to a wireless communication module of a medication delivery device to wirelessly communicate with the medication delivery device over an unencrypted wireless communication channel, wherein the medication delivery device comprises (a) a housing comprising a reservoir sized sufficiently to hold medication, (b) a dose button being rotatable relative to the housing to select a dose size of the medication for an injection, and (c) a controller in communication with the wireless communication module; and
receive, from the medication delivery device using the wireless communication channel, encrypted data over the unencrypted wireless communication channel, wherein the encrypted data comprises injection event information generated by the medication delivery device.

30. The computerized method of claim 29, further comprising decoding, from a two-dimensional barcode on the medication delivery device, encryption data that can be used to decrypt the encrypted data.

31. The computerized method of claim 30, wherein the encryption data comprises a private encryption key.

32. The computerized method of claim 29, wherein receiving the encrypted data further comprises receiving an unencrypted identifier for the medication delivery device.

33. The computerized method of claim 29, further comprising wirelessly receiving an advertising packet to advertise the presence of the medication delivery device for connection.

34. The computerized method of claim 33, wherein the advertising packet is encrypted, and the method further comprises decrypting the advertising packet.

35. The computerized method of claim 29, wherein connecting to the medication delivery device to wirelessly communicate with the medication delivery device over the unencrypted wireless communication channel comprises connecting to the medication delivery device using an unencrypted Bluetooth communication channel.

36. The medication delivery device of claim 17, comprising a medication disposed within said reservoir.
